(19) United States
(12) Patent Application Publication
(10) Pub. No.: US 2017/0171677 A1
(43) Pub. Date: Jun. 15, 2017

(54) TINNITUS TREATMENT SYSTEMS AND METHODS

(71) Applicant: Turtle Beach Corporation, San Diego, CA (US)

(72) Inventors: Elwood Grant Norris, Poway, CA (US); Brian Alan Kappus, San Diego, CA (US); James Arthur Bames, Las Vegas, NV (US); Sara Louise Madison, San Diego, CA (US); Ritvik Prakash Mehta, Solana Beach, CA (US)

(73) Assignee: Turtle Beach Corporation, San Diego, CA (US)

(21) Appl. No.: 14/966,996

(22) Filed: Dec. 11, 2015

(51) Int. Cl. H04R 25/00; A61B 5/12; A61N 7/00

(52) U.S. Cl. CPC H04R 25/75; A61N 7/00; A61B 5/128; A61F 2011/145

(57) ABSTRACT

A tinnitus treatment device can include an ultrasonic signal generator; an amplifier coupled to the ultrasonic signal generator; and an ultrasonic emitter coupled to an audio generating apparatus configured to output an audio modulated ultrasonic carrier signal into the air; wherein parameters of the tinnitus treatment device are configured to deliver tinnitus masking audio content.

### TECHNICAL FIELD

The disclosed technology relates generally to audio content, and more particularly, some embodiments relate to systems and methods for masking or abating tinnitus symptoms with ultrasonic emitters.

### BACKGROUND OF THE INVENTION

According to the National Institutes of Health, of adults ages 65 and older in the United States, 12.3 percent of men and nearly 14 percent of women are affected by tinnitus. Tinnitus is the sound heard in the head but has no external source. For some it manifests itself as a ringing in the ears, but is not limited to this as others may hear buzzing, hissing, humming, roaring or other sounds that are not really there. Tinnitus can be caused by problems in the patient's outer, middle or inner ear, or it can be caused by damaged auditory nerves or damage to the brain's auditory pathways. In fact, most tinnitus is believed to be caused by hearing loss at the cochlea or cochlear nerve level. More particularly, it is believed that there is an association between higher frequency hearing loss and tinnitus.

Another form of tinnitus, referred to as objective tinnitus, may be a result of sound heard by actual conditions within the patient's body such as, for example, turbulent blood flow in stiffened arteries, or other blood vessel damage. There is not believed to be any FDA-approved drug to treat tinnitus, or any supplements or herbs that have been proven to be more effective than a placebo in controlled clinical trials although this is a continuing area of medical research. Professionals have tried approaches such as cognitive behavioral therapy and tinnitus retraining therapy to help patients manage tinnitus symptoms. Cognitive behavioral therapy is akin to a psychological treatment that does not remove the symptoms, but helps patients manage the symptoms. Tinnitus retraining therapy uses a sound generator to generate low-level noise or other sounds in the patient's ear. Ideally, the sounds match the volume and frequency of the patient's tinnitus. Others have employed masking devices, which generate white, pink, red and other noise intended to reduce the perception of tinnitus or mask the ringing sound.

Prior attempts to counter or mask tinnitus have included traditional audio systems employed to focus on masking the perceived sound and systems creating pulsed ultrasound noise to deliver vibrations to the subject. Traditional audio solutions include a number of commercial systems as stand alone devices or as devices to provide therapy through a person's existing hearing aids.

While there is disagreement about the source of tinnitus (whether it is caused by the ear or the brain), most researchers agree that tinnitus and hearing loss are linked. Some deaf individuals also complain of bothersome tinnitus. Conventional tinnitus maskers are not very effective with those persons who have profound hearing loss.

Parametric audio reproduction systems produce sound by heterodyning two acoustic signals in a non-linear process that occurs in a medium such as air. The acoustic signals are typically in the ultrasound frequency range. The non-linearity of the medium results in acoustic signals produced by the medium that are the sum and difference of the acoustic signals. Thus, two ultrasound signals that are separated in frequency can result in a difference tone that is within the 60 Hz to 20,000 Hz range of human hearing.

### BRIEF SUMMARY OF EMBODIMENTS

According to various embodiments of the disclosed technology ultrasonic audio systems are provided as a manner of addressing the symptoms of tinnitus. Ultrasonic audio systems and methods such as those described herein, and including those used in the tests that are documented herein, can provide improved clarity and comprehension for someone with mild to severe hearing loss as compared to conventional audio systems, and can provide improved high-frequency hearing assistance as compared to conventional hearing aids. Additionally, in-ear ultrasonic audio systems and methods described herein can provide improved low-frequency response (e.g. below 400 or 500 Hz) as compared to conventional hearing aids.

Just as clarity and comprehension is important to those with hearing loss; ultrasonic audio devices such as those described herein that deliver high clarity provide better tinnitus therapy than traditional audio which is traditionally provided by conventional headphones, earbuds or hearing aids. Additionally, the ultrasonic audio systems can deliver directed audio targeting a listener and provide therapy to the listener without interfering with other persons nearby. This is due to the directional nature of the audio delivery that can be achieved with ultrasonic audio systems. Conventional freestanding audio loudspeakers (other than headphones, earbuds or hearing aids) cannot provide this personal sound therapy, as the sound from conventional audio loudspeakers tends to travel throughout the room or other listening environment. Indeed, ultrasonic audio systems such as those described herein can be configured to target each ear of a listener from a distance to provide therapy without interrupting other sounds in the environment. For example, ultrasonic audio treatment can be delivered to and provide therapy to a listener in bed without distracting his or her sleep partner and without the need for cumbersome headphones. A similar advantage can be achieved in other environments such as at work or in a room or other listening area where other people are present enjoying conversation, television, music, or quiet time.

In some embodiments, the ultrasonic tinnitus treatment system can also be configured as a hearing aid. Accordingly, in-ear ultrasonic audio systems can be used for the dual purpose of compensating for the hearing loss of a listener as well as providing tinnitus treatment. Embodiments of such a dual-purpose system can be implemented to achieve one or more of the various features described above.

In still further embodiments, the ultrasonic audio system can be configured to address the symptoms of tinnitus by stimulating the supporting cells and membranes surrounding the cilia of the inner ear. For example, systems can be configured to stimulate the basilar membrane with an ultrasonic signal, or with audio content demodulated from the ultrasonic signal. The ultrasonic signal can be delivered through bone conduction, by transmission through the eardrum and the ossicles (i.e., Malleus, Incus and Stapes) or otherwise delivered to the inner ear. In other embodiments the improved audio content (higher clarity, improve frequency response) can be delivered by demodulation in the air, or by demodulation within the inner ear. In some embodiments, the frequency of the ultrasonic carrier can be chosen to be at or near the resonant frequency of the basilar membrane to provide basilar membrane stimulation.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The present invention, in accordance with one or more various embodiments, is described in detail with reference to the accompanying figures. The drawings are provided for purposes of illustration only and merely depict typical or example embodiments of the invention. These drawings are provided to facilitate the reader's understanding of the systems and methods described herein, and shall not be considered limiting of the breadth, scope, or applicability of the claimed invention.

Some of the figures included herein illustrate various embodiments of the invention from different viewing angles. Although the accompanying descriptive text may refer to elements depicted therein as being on the “top,” “bottom” or “side” of an apparatus, such references are merely descriptive and do not imply or require that the invention be implemented or used in a particular spatial orientation unless explicitly stated otherwise.

FIG. 1 is a diagram illustrating an ultrasonic sound system suitable for use with the emitter technology described herein.

FIG. 2 is a diagram illustrating another example of a signal processing system that is suitable for use with the emitter technology described herein.

FIG. 3 is a diagram illustrating an example electrostatic emitter for use in an in-ear emitter/transducer in accordance with one embodiment of the technology described herein.

FIG. 4A is a top view of an example piezoelectric transducer with an impedance matching element in accordance with one embodiment of the technology described herein.

FIG. 4B is a cross-sectional view of the example piezoelectric transducer with an impedance matching element of FIG. 4A.

FIG. 5A is a cross-sectional view of a piezo crystal transducer with an impedance matching element in accordance with one embodiment of the technology described herein.

FIG. 5B is a cross-sectional view of a piezoelectric stack transducer with an impedance matching element in accordance with one embodiment of the technology described herein.

FIG. 6 is a diagram illustrating an example of the output sound pressure level (OSPL) of a conventional hearing aid.

FIG. 7 is a diagram illustrating an example of the output sound pressure level (OSPL) of an ultrasonic emitter made in accordance with embodiments of the techniques disclosed herein.

FIG. 8 is a diagram illustrating an ultrasonic tinnitus treatment system in accordance with one embodiment of the systems and methods described herein.

FIG. 9 is a diagram illustrating an ultrasonic tinnitus treatment system in accordance with another embodiment of the systems and methods described herein.

FIG. 10 is a cutaway diagram of ultrasonic in-ear headphone housings/enclosures in accordance with one embodiment.

FIG. 11 is a diagram illustrating an example ultrasonic headphone treatment system in accordance with another embodiment of the systems and methods described herein.

FIG. 12 is a cutaway diagram illustrating an example ultrasonic emitter configuration of an example ultrasonic headphone in accordance with one embodiment of the technology described herein.

FIG. 13 is an operational flow diagram illustrating a process for determining an ultrasonic therapy for a tinnitus sufferer in accordance with one embodiment of the systems and methods described herein.

FIG. 14 is an operational flow diagram illustrating a process for ultrasonic therapy for a tinnitus sufferer in accordance with one embodiment of the systems and methods described herein.

FIG. 15 is an operational flow diagram illustrating an example process for administering the treatment in accordance with an embodiment in which the subject's hearing response profile is also determined.

FIG. 16 is an operational flow diagram illustrating another process for determining and delivering an ultrasonic therapy for a tinnitus sufferer in accordance with one embodiment of the systems and methods described herein.

FIG. 17 is a diagram illustrating an example of an ultrasonic audio system for tinnitus therapy in accordance with one embodiment of the systems and methods described herein.

The figures are not intended to be exhaustive or to limit the invention to the precise form disclosed. It should be understood that the invention can be practiced with modification and alteration, and that the invention be limited only by the claims and the equivalents thereof.

### DESCRIPTION

Embodiments of the systems and methods described herein provide a HyperSound audio system (HSS) or other ultrasonic audio system for a variety of different applications. Embodiments of the technology described herein include ultrasonic emitters, ultrasonic headphones or small ultrasonic transducers at or in the ear. Certain embodiments provide an ultrasonic audio system for the treatment of tinnitus or other like symptoms or maladies. In particular, in various embodiments ultrasonic signals can be generated and directed from ultrasonic emitters toward a listener having tinnitus symptoms to help alleviate those symptoms. The ultrasonic emitters can be placed in the room with the listener, mounted as headphones or ear buds, or otherwise placed at or in the ear. The ultrasonic signals can be unmodulated ultrasonic signals or ultrasonic signals modulated with audio content. The modulated audio content can include one or more audio tones, white noise, brown noise, gray noise, pink noise or other audio artifacts, or it can include programmatic content such as music, narratives, and so on. The ultrasonic signals can have a range of carrier frequencies, as discussed herein.

One ultrasonic emitter can be used to provide therapy, or multiple ultrasonic emitters can be used to direct ultrasonic signals at the tinnitus sufferer or at or in the ear of such person. Because ultrasonic signals tend to be highly directional, any ultrasonic wave therapy or sound or other audio content modulated onto the ultrasonic carriers can be selectively directed at the intended user and the system can accordingly be configured to create a personal therapeutic or listening environment. That is, the system can be configured to take advantage of the directional nature of ultrasonic signals such that a therapeutic ultrasonic wave or audio content can be directed at and heard only by the intended listener (or others in the path of the signals). Other embodiments deliver a personal therapy through headphones or at or in the ear using a small ultrasonic transducer.

As noted, the symptoms of tinnitus can be caused by a number of factors. One such factor is a loss of hearing. With many individuals, hearing loss begins with or is more pronounced at higher frequencies. Indeed, for experiencing hearing loss, many of those individuals have excellent hearing at the lower and mid-frequency ranges while having significantly reduced or no hearing at the higher frequencies. Audio content delivered by ultrasonic carriers in accordance with the systems and methods described herein can tend to overcome certain high-frequency hearing loss delivering increased comprehension and clarity at normal listening volumes (e.g., 30 dB, 40 dB, 50 dB, 60-65 dB, 70 dB, 80 dB, or anywhere within that range. Traditional hearing aids attempt to overcome hearing loss by increasing volumes at the affected hearing loss frequencies. Some conventional hearing aids can provide output at greater than 100 dB and as much as 120 dB or more. Such levels are often required for patients with hearing disabilities based on the quality and frequency response of the conventional hearing aid. However, due to the clarity and frequency response of ultrasonic audio systems, lower sound pressure levels, as noted above, can be used to achieve similar or improved results (including comprehension) to conventional hearing aids.

In studies conducted by or on behalf of the inventors, individuals exhibited improved hearing at high frequencies when listening to ultrasonic audio delivered in accordance with the systems and methods described herein as compared with conventional audio speakers (e.g., speakers that rely on back-and-forth motion of a cone, ribbon, membrane or other driver to create sound waves in the range of normal hearing) without increasing the volume.

Because individuals with high-frequency hearing loss can typically hear high-frequency audio delivered through such ultrasonic systems, the use of the systems can help to overcome (by retraining/masking) one of the causes (or associated malady) of the tinnitus symptoms (inability to hear high frequencies). Likewise ultrasonic systems can provide those with high-frequency hearing loss with clearer and more precise masking than conventional audio sources. Accordingly, audio content delivered by such ultrasonic systems can have a greater therapeutic effect on the subject than conventional masking audio delivered by conventional loudspeaker systems or by existing hearing aids that are programmed to deliver masking audio.

The ultrasonic systems described herein are systems using ultrasonic technology designed to target a specific listening area or provide a personal therapeutic relief such personal relief can be delivered using freestanding ultrasonic emitters with sufficient directional quality to direct the audio at the intended listener or through ultrasonic headphones or earpieces. These systems can provide ultrasonic wave therapy to a specific listener or these systems can electronically modulate audible therapy information onto ultrasonic waves. This audio content is carried in an air beam of silent ultrasound energy. If audio content is modulated onto the ultrasonic wave it is then demodulated in the air reproducing the audible tones such that, in some embodiments, the audio can be heard only by those in the targeted area or those receiving the energy. As noted, the ultrasonic energy can be delivered by in-room ultrasonic emitters or by at- or in-the-ear ultrasonic emitters in the case of ultrasonic headphones or small ultrasonic transducers at or in the ear.

Unlike a conventional speaker, sound is not created omni-directionally at the speaker (emitter) surface but is created along and within a highly directional air column. The high precision targeting of such systems significantly minimizes the levels of noise pollution in both open and confined spaces and anywhere else ambient noise is an issue. Such systems can be configured to cut through other ambient noise so the targeted area gets a clear high-fidelity audio content. Because this targeting also cuts through or reduces the perception of ambient noise, it offers the ability to provide therapy (commonly delivered alone at quiet times) in other environments.

A clinical study was performed at the direction of the inventors. This study was performed in a controlled audiology laboratory as a single-blinded, randomized cross over clinical study using adult subjects with hearing loss ranging from mild to severe degree. Testing was based upon speech tests including Consonant-Nucleus-Consonant or CNC in quiet and AzBio in noise to assess the effectiveness of the HSS System compared to conventional speakers at comparable volume levels.

Ten adult patients with mild to severe hearing loss with pure-tone average (PTA) of >30 dB and word discrimination scores of <80% in both ears were tested at one meter distance from the audio source. Significant gains in speech understanding were associated with the HSS versus conventional speaker for all test conditions at 70 dB. These studies show a marked improvement in sound clarity/increased high frequency output at lower volumes using standardized speech perception testing methods including. Participants in these studies experienced significantly greater sound clarity when listening to sound through the ultrasonic emitter system compared to the conventional audio speaker at 70 dB.

Of particular note is the improvement in clarity scores in the presence of background noise. The test results indicate that participants achieved sound clarity test scores of 38.2% correct on the AzBio Sentences test at 70 dB in a quiet environment with a standard deviation of ±33.4. This demonstrates an improvement over conventional speakers of greater than 3 times. At 70 dB in a noisy environment (noise condition of a mean of 42.6 db vs a mean of 38.2 db for the quiet condition), participants achieved sound clarity test scores of 42.6% correct on the AzBio Sentences test with a standard deviation of ±33.7. Median AZBio scores increased from 0.0% to 34.9% (p=0.008) in quiet, and from 1.8% to 51.6% in noise (p=0.008). These results show an improvement over conventional speakers of greater than five times.

On the CNC word test, at 70 dB in a quiet environment, participants scored 44.4% using the ultrasonic emitter system as compared to only 6.0% with conventional speakers. This represents an improvement of greater than seven times over conventional speakers. At 70 dB in a noisy environment, participants scored 56.5% with the ultrasonic emitter system as compared to only 15.4% with a conventional audio system. Median CNC whole word test scores increased from 0.0% to 54.0% (p=0.004) and median phoneme test scores from 4.0% to 63.4% (p=0.004).

Also, a study was performed by an accredited independent laboratory pursuant to ANSI S3.2-2009-American National Standard Method for Measuring the Intelligibility of Speech over Communication Systems. Testing of the HSS system demonstrated a mean ANSI S3.2-2099 MRT (Modified Rhyme Test) score of 91% while a conventional speaker device had a mean score of 79.2% demonstrating greater word list discrimination in noise amongst five normal hearing subjects listening to five recorded talkers. For each device pink noise at 68.7 dBA was employed with speech at 72-74 dBA for a signal to noise ration of approximately 6 dBA.

Reasons that participants experienced greater sound clarity with the ultrasonic emitter system, especially in the presence of background noise, may include one or more of the following characteristics of the HSS systems described herein: high precision targeting of sound, the superior transient response of ultrasonic audio and/or improved ear pathway response. Unlike a conventional audio speaker that emits sound omni-directionally from the speaker surface, the HSS creates sound along and within a highly directional air column. The high precision targeting of the HSS significantly minimizes the levels of ambient noise pollution so the targeted area gets a clear high-fidelity audible message. HSS delivers superior transient response important for clear messaging at or near the ear pathway for improved audio response.

Further experiments performed by the inventors indicate improved pure-tone threshold levels when utilizing HSS/ultrasonic in-ear devices versus conventional headphones. Compared to Telephonics TDH-39P conventional headphones and using an individual with sloping hearing loss to 90 dB, an HSS/ultrasonic in-ear device in accordance with one embodiment of the technology disclosed herein shows a 21 dB increase in sensitivity at 8 kHz. These tests were conducted in an audiologist soundroom using calibrated input.

Additionally, the inventors' experiments show that hearing aids employing such ultrasonic earpieces not only provide improved therapy for those with high frequency hearing loss at normal audio volumes but also can deliver therapy across a broad frequency range including low frequencies (e.g., down to 40 Hz).

Further experiments by the inventors show that audio-modulated ultrasound delivered to the ear offers improved clarity as compared to audio-modulated ultrasound that is interrupted by mylar shielding prior to delivery to the ear. This is indicative of improved ear pathway response.

A number of ultrasonic audio systems can be suitable for delivery of audio content for the treatment of tinnitus symptoms. A few examples of such ultrasonic systems are described herein. This description is followed by a description of the treatment process using these and other ultrasonic audio systems.

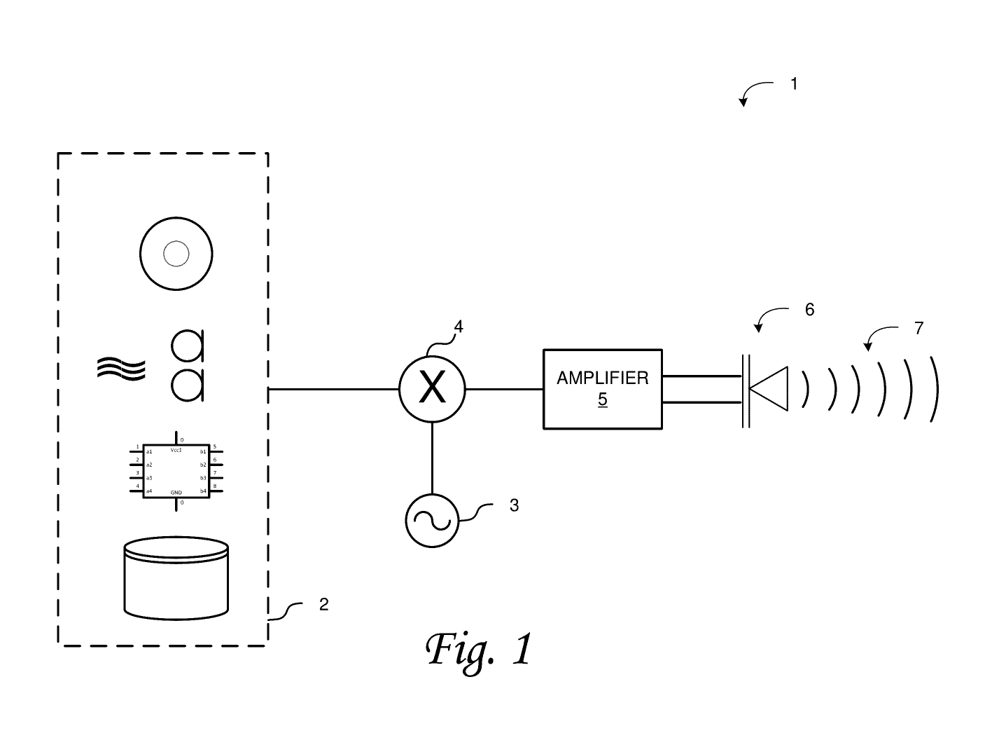

FIG. 1 is a diagram illustrating an ultrasonic sound system suitable for use in conjunction with the systems and methods described herein. In this exemplary ultrasonic system 1, audio content from an audio source 2, such as, for example, a microphone, memory, a data storage device, streaming media source, MP3, CD, DVD, set-top-box, or other audio source is received. The audio content may be decoded and converted from digital to analog form, depending on the source. The audio content received by the audio system 1 is modulated onto an ultrasonic carrier of frequency f1, using a modulator. The modulator typically includes a local oscillator 3 to generate the ultrasonic carrier signal, and multiplier 4 to modulate the audio signal on the carrier signal. The resultant signal is a double- or single-sideband signal with a carrier at frequency f1 and one or more side lobes. In some embodiments, the signal is a parametric ultrasonic wave or an HSS signal. In most cases, the modulation scheme used is amplitude modulation, or AM, although other modulation schemes can be used as well. Amplitude modulation can be achieved by multiplying the ultrasonic carrier by the information-carrying signal, which in this case is the audio signal. The spectrum of the modulated signal can have two sidebands, an upper and a lower side band, which are symmetric with respect to the carrier frequency, and the carrier itself.

The modulated ultrasonic signal is provided to the transducer 6, which launches the ultrasonic signal into the air creating ultrasonic wave 7. When played back through the transducer at a sufficiently high sound pressure level, due to nonlinear behavior of the air through which it is ‘played’ or transmitted, the carrier in the signal mixes with the sideband(s) to demodulate the signal and reproduce the audio content. This is sometimes referred to as self-demodulation. Thus, even for single-sideband implementations, the carrier is included with the launched signal so that self-demodulation can take place.

Although the system illustrated in FIG. 1 uses a single transducer to launch a single channel of audio content, one of ordinary skill in the art after reading this description will understand how multiple mixers, amplifiers and transducers can be used to transmit multiple channels of audio using ultrasonic carriers. The ultrasonic transducers can be mounted in any desired location depending on the application.

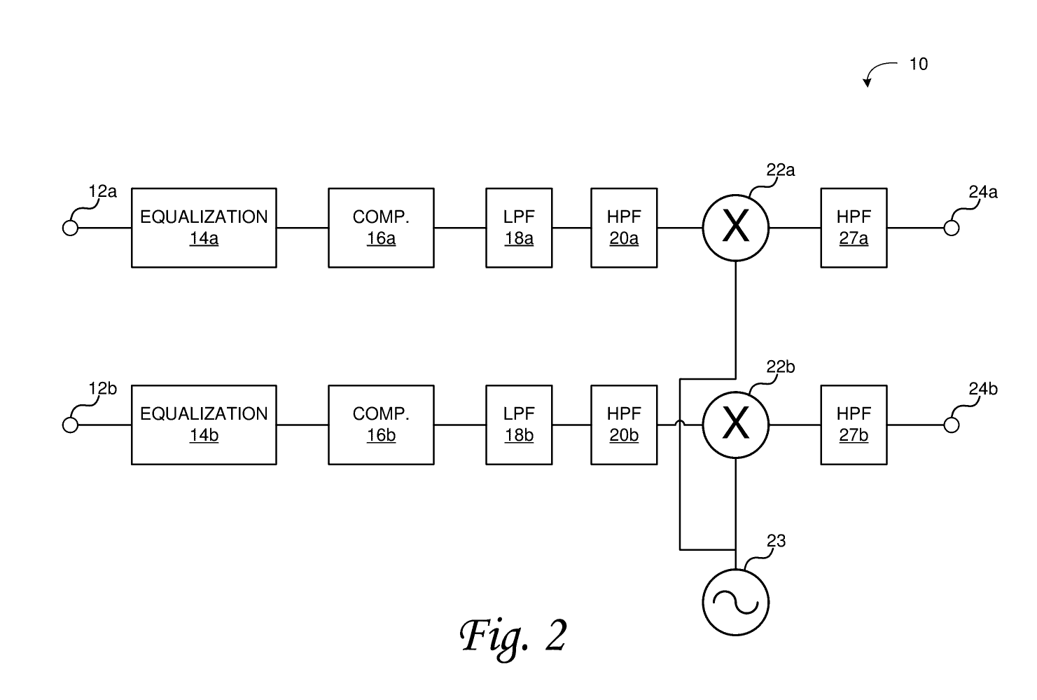

One example of a signal processing system 10 that is suitable for use with the technology described herein is illustrated schematically in FIG. 2. In this embodiment, various processing circuits or components are illustrated in the order (relative to the processing path of the signal) in which they are arranged according to one implementation. It is to be understood that the components of the processing circuit can vary, as can the order in which the input signal is processed by each circuit or component. Also, depending upon the embodiment, the processing system 10 can include more or fewer components or circuits than those shown.

Also, the example shown in FIG. 1 is optimized for use in processing two input and output channels (e.g., a “stereo” signal), with various components or circuits including substantially matching components for each channel of the signal. It will be understood by one of ordinary skill in the art after reading this description that the audio system can be implemented using a single channel (e.g., a “monaural” or “mono” signal), two channels (as illustrated in FIG. 2), or a greater number of channels.

Referring now to FIG. 2, the example signal processing system 10 can include audio inputs that can correspond to left 12a and right 12b channels of an audio input signal. Equalizing networks 14a, 14b can be included to provide equalization of the signal. The equalization networks can, for example, boost or suppress predetermined frequencies or frequency ranges to increase the benefit provided naturally by the emitter/inductor combination of the parametric emitter assembly. Such equalization can be adjusted to compensate for an individual user's hearing loss.

After the audio signals are equalized compressor circuits 16a, 16b can be included to compress the dynamic range of the incoming signal, effectively raising the amplitude of certain portions of the incoming signals and lowering the amplitude of certain other portions of the incoming signals. More particularly, compressor circuits 16a, 16b can be included to narrow the range of audio amplitudes. In one aspect, the compressors lessen the peak-to-peak amplitude of the input signals by a ratio of not less than about 2:1. Adjusting the input signals to a narrower range of amplitude can be done to minimize distortion, which is characteristic of the limited dynamic range of this class of modulation systems. In other embodiments, the equalizing networks 14a, 14b can be provided after compressors 16a, 16b, to equalize the signals after compression.

Low pass filter circuits 18a, 18b can be included to provide a cutoff of high portions of the signal, and high pass filter circuits 20a, 20b providing a cutoff of low portions of the audio signals. In one exemplary embodiment, low pass filters 18a, 18b are used to cut signals higher than about 15-20 kHz, and high pass filters 20a, 20b are used to cut signals lower than about 20-200 Hz.

The high pass filters 20a, 20b can be configured to eliminate low frequencies that, after modulation, would result in deviation of carrier frequency (e.g., those portions of the modulated signal that are closest to the carrier frequency). Also, some low frequencies are difficult for the system to reproduce efficiently and as a result, much energy can be wasted trying to reproduce these frequencies. Therefore, high pass filters 20a, 20b can be configured to cut out these frequencies.

The low pass filters 18a, 18b can be configured to eliminate higher frequencies that, after modulation, could result in the creation of an audible beat signal with the carrier. By way of example, if a low pass filter cuts frequencies above 15 kHz, and the carrier frequency is approximately 44 kHz, the difference signal will not be lower than around 29 kHz, which is still outside of the audible range for humans. However, if frequencies as high as 25 kHz were allowed to pass the filter circuit, the difference signal generated could be in the range of 19 kHz, which is within the range of some human hearing.

In the example system 10, after passing through the low pass and high pass filters, the audio signals are modulated by modulators 22a, 22b. Modulators 22a, 22b, mix or combine the audio signals with a carrier signal generated by oscillator 23. For example, in some embodiments a single oscillator (which in one embodiment is driven at a selected frequency of 40 kHz to 150 kHz, which range corresponds to readily available crystals that can be used in the oscillator) is used to drive both modulators 22a, 22b. By utilizing a single oscillator for multiple modulators, an identical carrier frequency is provided to multiple channels being output at 24a, 24b from the modulators. Using the same carrier frequency for each channel lessens the risk that any audible beat frequencies may occur.

High-pass filters 27a, 27b can also be included after the modulation stage. High-pass filters 27a, 27b can be used to pass the modulated ultrasonic carrier signal and ensure that no audio frequencies enter the amplifier via outputs 24a, 24b. Accordingly, in some embodiments, high-pass filters 27a, 27b can be configured to filter out signals below about 25 kHz.

Any of a number of ultrasonic emitters can be used with the technology disclosed herein. A few examples of emitters and associated technology that can be used with the systems and methods disclosed herein include those emitters and associated technology disclosed in U.S. Pat. No. 8,718,297 to Norris, titled Parametric Transducer and Related Methods, and U.S. Pat. No. 9,002,043 to Norris, titled Parametric Transducer and Related Methods, which are each incorporated by reference herein in their entirety as if reproduced in full below. It will also be appreciated by those of ordinary skill in the art after reading this description how the technology can be implemented using other ultrasonic emitters and alternative driver circuitry. For example, an emitter can be configured in a size and shape for application as an in-ear emitter as described below.

As described herein, various embodiments can be configured to transmit one or more channels of audio using ultrasonic carriers. The transmission of audio using ultrasonic carriers can be used in a variety of different scenarios/contexts as will be described in greater detail below. For example, various embodiments may be utilized in or for implementing directed/targeted or isolated sound systems, headphones, hearing aids or other assistive hearing devices including such devices targeted to provide masking therapy to tinnitus sufferers.

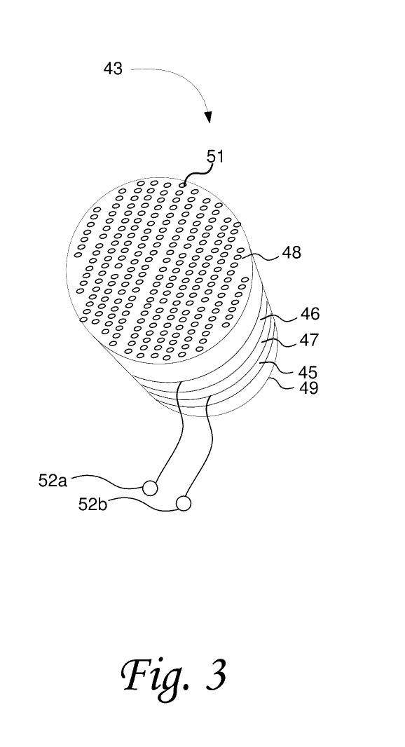

FIG. 3 illustrates an example configuration for an ultrasonic emitter that can be configured in an appropriate size and shape to form an in-ear emitter. The example emitter 43 shown in FIG. 3 includes one conductive surface 45, another conductive surface 46, an insulating layer 47 and a screen or mesh 48. In the illustrated example, conductive layer 45 is disposed on a backing plate 49. In various embodiments, backing plate 49 is a non-conductive backing plate and serves to insulate conductive surface 45 on the back side. For example, conductive surface 45 and backing plate 49 can be implemented as a metalized layer deposited on a non-conductive, or relatively low conductivity, substrate. As a further example, a plastic or other like substance can be used to form a textured backing plate substrate, which can be metalized. Such a substrate can be injection molded, machined or manufactured using other like techniques.

As a further example, conductive surface 45 and backing plate 49 can be implemented as a printed circuit board (or other like material) with a metalized layer deposited thereon. As another example, conductive surface 45 can be laminated or sputtered onto backing plate 49, or applied to backing plate 49 using various deposition techniques, including vapor or evaporative deposition, and thermal spray, to name a few. As yet another example, conductive layer 45 can be a metalized film.

Conductive surface 45 can be a continuous surface or it can have slots, holes, cut-outs of various shapes, or other non-conductive areas. Additionally, conductive surface 45 can be a smooth or substantially smooth surface, or it can be rough or pitted. For example, conductive surface 45 can be embossed, stamped, sanded, sand blasted, formed with pits or irregularities in the surface, deposited with a desired degree of ‘orange peel’ or otherwise provided with texture.

Conductive surface 45 need not be disposed on a dedicated backing plate 49. Instead, in some embodiments, conductive surface 45 can be deposited onto a member that provides another function, such as a member that is part of a speaker housing. Conductive surface 45 can also be deposited directly onto a wall or other location where the emitter is to be mounted, and so on.

Conductive surface 46 provides another pole of the emitter. Conductive surface can be implemented as a metalized film, wherein a metalized layer is deposited onto a film substrate (not separately illustrated). The substrate can be, for example, polypropylene, polyimide, polyethylene terephthalate (PET), biaxially-oriented polyethylene terephthalate (e.g., Mylar, Melinex or Hostaphan), Kapton, or other substrate. In some embodiments, the substrate has low conductivity and, when positioned so that the substrate is between the conductive surfaces of layers 45 and 46, acts as an insulator between conductive surface 45 and conductive surface 46. In other embodiments, there is no non-conductive substrate, and conductive surface 46 is a sheet of conductive material. Graphene or other like conductive materials can be used for conductive surface 46, whether with or without a substrate.

In addition, in some embodiments, conductive surface 46 (and its insulating substrate where included) is separated from conductive surface 45 by an insulating layer 47. Insulating layer 47 can be made, for example, using PET, axially or biaxially-oriented polyethylene terephthalate, polypropylene, polyimide, or other insulative film or material.

To drive the emitter 43 with enough power to get sufficient ultrasonic pressure level, arcing can occur where the spacing between conductive surface 46 and conductive surface 45 is too thin. However, where the spacing is too thick, the emitter 43 will not achieve resonance, nor will it be sensitive enough. In one embodiment, insulating layer 47 is a layer of about 0.92 mil in thickness. In some embodiments, insulating layer 47 is a layer from about 0.90 to about 1 mil in thickness. In further embodiments, insulating layer 47 is a layer from about 0.75 to about 1.2 mil in thickness. In still further embodiments, insulating layer 47 is as thin as about 0.33 or 0.25 mil in thickness. Other thicknesses can be used, and in some embodiments a separate insulating layer 47 is not provided. For example, some embodiments rely on an insulating substrate of conductive layer 46 (e.g., the base layer in the case of a metalized film) to provide insulation between conductive surfaces 45 and 46. One benefit of including an insulating layer 47 is that it can allow a greater level of bias voltage to be applied across the first and second conductive surfaces 45, 46 without arcing. When considering the insulating properties of the materials between the two conductive surfaces 45, 46, one should consider the insulating value of layer 47, if included, and the insulating value of the substrate, if any, on which conductive layer 46 is deposited.

A grating 48 can be included on top of the stack, although it is not necessary. Grating 48 can be made of a conductive or non-conductive material. Because grating 48 is in contact in some embodiments with the conductive surface 46, grating 48 can be made using a non-conductive material to shield users from the bias voltage present on conductive surface 46. Grating 48 can include holes 51, slots or other openings. These openings can be uniform, or they can vary across the area, and they can be thru-openings extending from one surface of grating 48 to the other. Grating 48 can be of various thicknesses. It should be noted that metal mesh material can be also used to effectuate shielding, for example, 165 thread-per-inch metal mesh having a 2 mil wire diameter. In order to be electrically isolated from conductive surface 46, spacing can be provided by way of a plastic frame. The metal mesh can be glued or otherwise adhesively attached to the plastic frame under tension so as to be sufficiently structurally strong to prevent being pushed into conductive surface 46.

Electrical contacts 52a, 52b are used to couple the modulated ultrasonic carrier signal into the emitter 43. The emitter 43 can be made to just about any dimension or shape. As illustrated in FIG. 3, emitter 43 is circular. In another application, the emitter is 1 cm long and 1 cm wide, although other dimensions, both larger and smaller are possible. Practical ranges of length and width can be similar lengths and widths of conventional in-ear speaker or hearing devices. Greater emitter area can lead to a greater sound output, but may also require higher bias voltages. It should be noted that with regard to this and other embodiments described and/or contemplated herein, an emitter may be configured in a variety of shapes as well as dimensions.

An electrostatic emitter can be optimized by adjusting one or more characteristics, such as but not limited to thickness and/or curvature in order to achieve impedance matching. In this example, conductive layer 46 may be optimized accordingly. Also, an intermediary material, such as aerogel, foam, or other appropriate material can be utilized proximate to but not touching conductive layer 46. For example, such a material can be disposed between conductive layer 46 and grating 48 (if a grating is used) or simply above conductive layer 46.

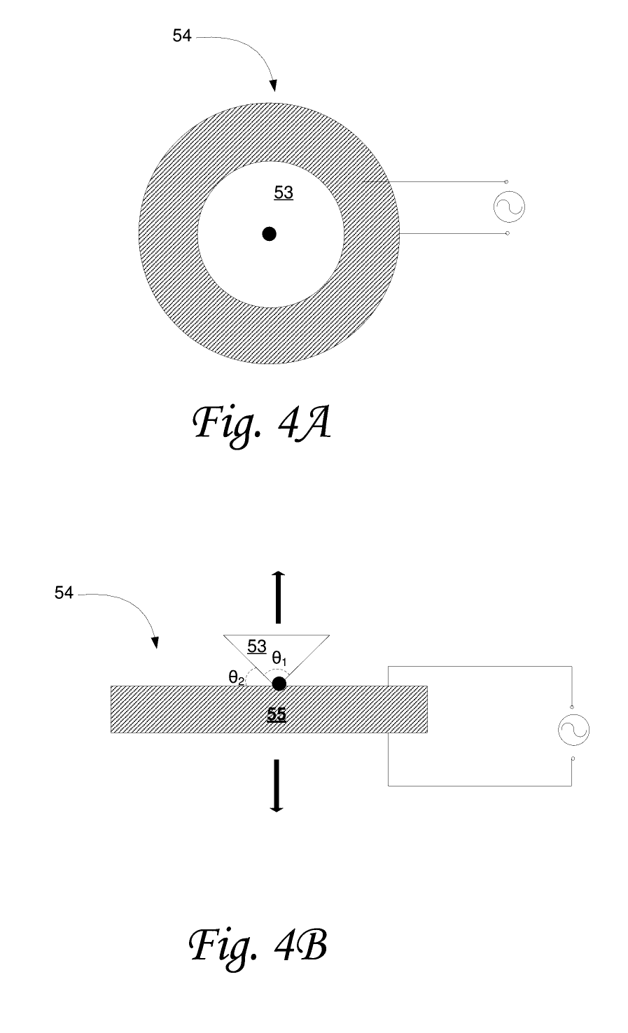

FIGS. 4A and 4B illustrate top and cross-sectional views, respectively, of another example emitter 54. In this example, the emitter 54 may be a piezoelectric transducer. That is, the emitter 54 may be made up of a piezoelectric or piezoceramic element 55. Similar to emitter 43 of FIG. 3, a signal may be applied to the emitter 54. However, piezoelectric or piezoceramic element 55, in this case, may expand and contract (rather than flex and bend) in order to launch an ultrasonic signal. That is and for example, when an appropriate electric field is placed across a thickness of piezoelectric element 55, piezoelectric element 55 can expand in thickness along its axis of polarization and contract in a transverse direction perpendicular to the axis of polarization and vice versa (when the field is reversed). It should be noted that piezoelectric or piezoceramic element 55 is configured such that it is resonant at the ultrasonic carrier frequency.

In this embodiment, an impedance matching element 53 may be utilized to optimize the listening experience by matching the impedance of the emitter 54 to that of, e.g., the ear canal (e.g., air within the ear canal or the outer ear proximate to the ear canal) of the listener. In this example, impedance matching element 52 may be a cone, but in other embodiments may be, e.g., aerogel, foam, or other material(s) or element(s) that can be utilized for impedance matching. For example, impedance matching element 53 may be tailored to or otherwise optimized for each user. In some embodiments, one or more impedance-relevant/related measurements can be made of a user's ear canal and the matching element 53 tailored to his/her ear. Generally, the impedance of a closed volume, such as a tubular space can be defined as the ratio between the effective sound pressure and the volume velocity, where the volume velocity can refer to the volume displacement times angular frequency. Other measurements/definitions of the in-ear impedance to be matched may be utilized/considered in accordance with various embodiments. For example, in some embodiments impedance may be measured at differing reference planes (at the entrance of the ear canal, some distance into the ear canal, etc.), and may or may not include the impedance of the eardrum plus the compliance of the flesh in the inner part of the ear canal.

In order to achieve the proper impedance matching, geometric parameters of the impedance matching element 53 can be tailored to meet the desired impedance matching characteristics. For example, one or more of the angles of the conical region of impedance matching cone (θ₁) and the angle of the conical region of impedance matching element 53 relative to the piezoelectric element 55 (θ₂) may be adjusted. The impedance matching element 53 may also be adjusted with regard to its thickness. For example, the walls of impedance matching element 53 may be thickened or thinned depending on the relevant impedance of the ear canal. Moreover, the walls of impedance matching element 53 may have a gradient thickness, and they be curved or otherwise, non-straight walls. Further still, impedance matching element 53 may be tailored with respect to overall size (e.g., height and diameter), weight, location relative to the piezoelectric element 55, etc.

A modulated ultrasonic signal can be provided to the piezoelectric element 55, such that in conjunction with impedance matching element 53, an ultrasonic signal is launched into the ear or ear canal, creating an ultrasonic wave. Due to the nonlinear behavior of the air within the ear canal through which it is ‘played’ or transmitted, the carrier in the signal mixes with the sideband(s) to demodulate the signal and reproduce the audio content within the ear canal. It should be noted that the inner ear is also nonlinear, and sound may be made/perceived within the ear, and not just in the ear canal.

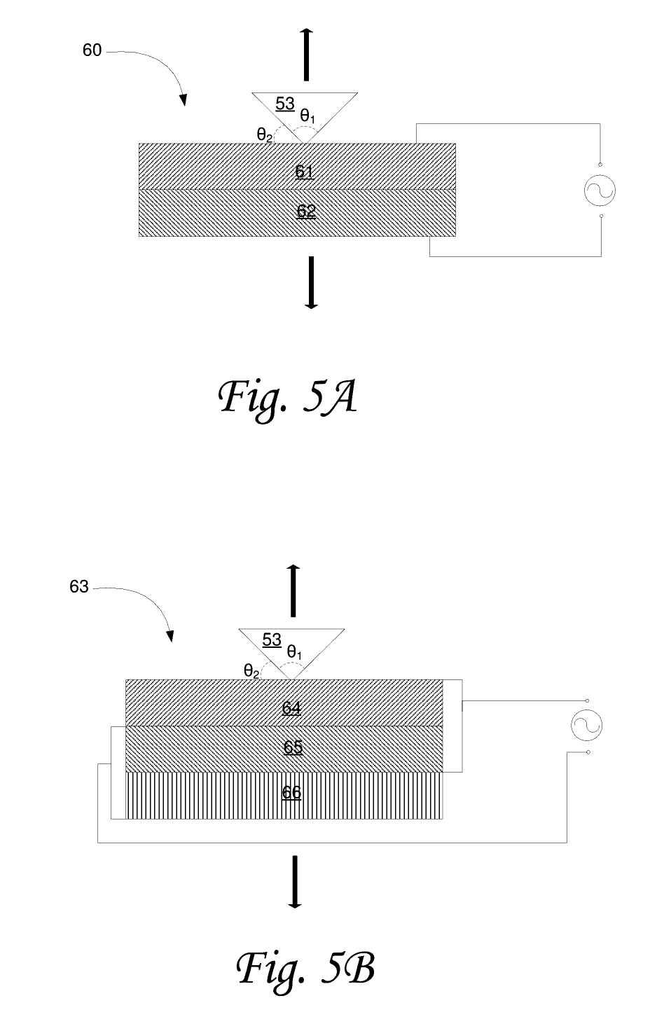

FIG. 5A illustrates another example emitter 60. In this example, the emitter 60 may be a bimorph emitter or transducer comprising two piezoelectric elements 61 and 62. Piezoelectric elements 61 and 62 may be oriented such that application of a signal causes piezoelectric elements 61 and 62 to expand or contract in concert with one another, and in conjunction with impedance matching element 53, effectuate launching of an ultrasonic signal into an ear or an ear canal.

It should be further noted that the natural frequency of the emitter may be approximately 85 kHz or higher to avoid audible sub-harmonics. Ideally, there can be a sufficient number of layers so that the (electrical) impedance is low enough to produce sufficient output with battery-voltages (˜1.35V). Higher voltages can be produced in the device in accordance with other embodiments. FIG. 5B illustrates yet another example emitter 63, where emitter 63 is a piezoelectric stack emitter including piezoelectric elements 64, 65, and 66. In this example, it should be understood that piezoelectric elements 64, 65, and 66 may be metalized allowing for the electrical connections illustrated in FIG. 5B to be made, which in turn, allow for synchronized expansion and contraction.

Various types of piezoelectric or piezoceramic materials/crystals may be utilized in accordance with various embodiments, including, e.g., barium titanate, lead zirconium titanate, gallium orthophosphate, langasite, lithium niobate, sodium tungstate, etc. Moreover, emitters made from such materials may also be adapted or configured with respect to, e.g., their shape and size, to achieve a desired response.

Studies have shown that, given the same audio volume, HSS can provide improved clarity and/or intelligibility compared to regular non-ultrasound audio. That is, various embodiments can provide the same or better clarity and/or intelligibility with less output (i.e., sound pressure level). Moreover, even if the output is increased, feedback associated with conventional hearing aids is reduced or even avoided. For example, conventional hearing assistive devices may be configured to provide amplification/gain resulting in audio transmission at approximately 125 dB, whereas the in-ear ultrasonic transducer device can provide the same or better clarity/intelligibility at only 80 db.

Additionally, due to the highly directional nature of ultrasonic audio signals, feedback can be reduced or virtually eliminated at operating levels. Because the audio demodulated from the ultrasonic signal is directed in the ear canal, little or no sound reflects back to the microphone and feedback can be avoided.

It should be noted that various driver circuits can be used to drive the emitters disclosed herein. In order to achieve reduced size/footprint of the in-ear ultrasonic transducer device, the driver circuit may be provided in the same housing or assembly as the emitter.

Typically, a modulated signal from a signal processing system is electronically coupled to an amplifier (as illustrated in FIG. 1). The amplifier can be part of, and in the same housing or enclosure as driver circuit. After amplification, the signal is delivered to inputs of the driver circuit. In the embodiments described herein, the emitter assembly includes an emitter that can be operable at ultrasonic frequencies.

In an electrostatic ultrasonic emitter, for example, a bias voltage can be applied to provide bias to the emitter. Ideally, the bias voltage used is approximately twice (or greater) the reverse bias that the emitter is expected to take on. This is to ensure that bias voltage is sufficient to pull the emitter out of a reverse bias state. In one embodiment, the bias voltage is on the order of 300-450 Volts, although voltages in other ranges can be used. For example, 350 Volts can be used. For ultrasonic emitters, bias voltages are typically in the range of a few hundred to several hundred volts.

The use of a step-up transformer also provides additional advantages to the present system. Because the transformer “steps-up” from the direction of the amplifier to the emitter, it necessarily “steps-down” from the direction of the emitter to the amplifier. Thus, any negative feedback that might otherwise travel from the inductor/emitter pair to the amplifier is reduced by the step-down process, thus minimizing the effect of any such event on the amplifier and the system in general (in particular, changes in the inductor/emitter pair that might affect the impedance load experienced by the amplifier are reduced).

For crystal and piezoelectric stack (including bimorphs) emitters 54 of FIGS. 4A and 4B, and PVDF emitters, it should be noted that no transformer/transductor is necessarily needed, nor is any bias voltage required. Rather, a high frequency amplifier may be used, such as a delta-sigma audio power amplifier.

In accordance with some embodiments, various technologies described herein can be applied to hearing aids or other assistive listening devices. For example, demodulation of an audio-encoded ultrasonic carrier signal can be accomplished within the listener's inner ear, taking into account impedance which can be matched with an impedance matching element and/or by optimizing a vibrating film to achieve impedance matching. Additionally, a hearing response profile of a listener can be determined, and audio content can be adjusted to at least partially compensate for the listener's hearing response profile. Again, the use of a parametric ultrasonic wave or a HSS signal in accordance with various embodiments holds particular advantages over conventional assistive hearing devices. That is, various embodiments, through the use of ultrasonics, may be configured to provide a perfect or at least near-perfect transient response, which can improve clarity, as opposed to conventional audio systems that can experience various types and/or varying amounts of distortion due to, e.g., the mass and/or resonance of drivers, enclosures, delay, etc. Moreover, conventional hearing aid devices amplify any and all sound, whereas various embodiments need not.

Various embodiments may also be utilized in the context of audio sensing or detection. For example, various embodiments may be utilized to detect otoacoustic emissions. That is, otoacoustic emissions are a low-level sound emitted by the cochlea (whether spontaneously or by way of some type of auditory stimulus). Such otoacoustic emissions may be used to test, e.g., the hearing capabilities of a newborn baby, diagnosis or certain auditory dysfunction, such as tinnitus. Thus, the increased sensitivity and impedance matching achieved in accordance with various embodiments can also achieve more precise or accurate diagnoses and testing.

Generally, ear pieces must be placed far within the ear canal to form a seal with the ear canal via some form of malleable foam or other material. While this aids in combating leaking sound/passive noise cancellation and assists with bass response, many users find such in-ear devices to be uncomfortable, as well as dangerous in certain circumstances as all or much of the ambient noise/sound is blocked. Accordingly, various embodiments of the technology disclosed herein may employ venting or some ‘open’ implementation, e.g., a housing having an air gap or vents, although other embodiments may be implemented in a sealed configuration as well. However, and (unexpectedly) unlike conventional devices that lose low frequency response in vented or open implementations, the in-ear ultrasonic transducer device, unlike conventional speakers, can provide improved low frequency/bass response even in a vented or open implementation.

As alluded to above, and in accordance with various embodiments, the use of ultrasonic emitters in place of or in addition to conventional speakers can achieve highly directional audio transmission. That is, sound may be optimally directed within a user's ear canal for better audio perception, as well as lessening or negating the escape/leaking of sound without being uncomfortable or dangerous. Moreover, demodulation could occur within the inner ear and, therefore, bypass some forms of age-associated or other forms of hearing loss.

Before describing further applications, embodiments and features of the technology disclosed herein, it is useful to describe some of the performance characteristics that can be achieved by embodiments of the disclosed technology as these performance characteristics can contribute to the efficacy of the treatments.

Tinnitus therapy is generally associated with low volume background sounds, which are challenging to hear for those with hearing loss, especially high frequency hearing loss. The technology described herein can be configured in some embodiments to deliver therapy that can be heard and comprehended at normal listening volumes by those with hearing loss. For example, systems and methods can be implemented to deliver therapy to a listener having a defined level of hearing loss, enabling a listener to hear the audio while providing only 80 dB sound pressure level to the listener. In contrast, conventional hearing aids for the same listener may need to produce as much as 120 dB SPL to enable the listener to hear the audio delivered by the hearing aid.

Additionally, although many conventional hearing aids tout a frequency response of up to 8 kHz to 10 kHz, most typically begin to roll off significantly at around 4 kHz. Accordingly, these conventional hearing aids, even a volumes at approximately hundred 20 dB SPL cannot provide sufficient levels at frequencies above 4 kHz to be clinically useful.

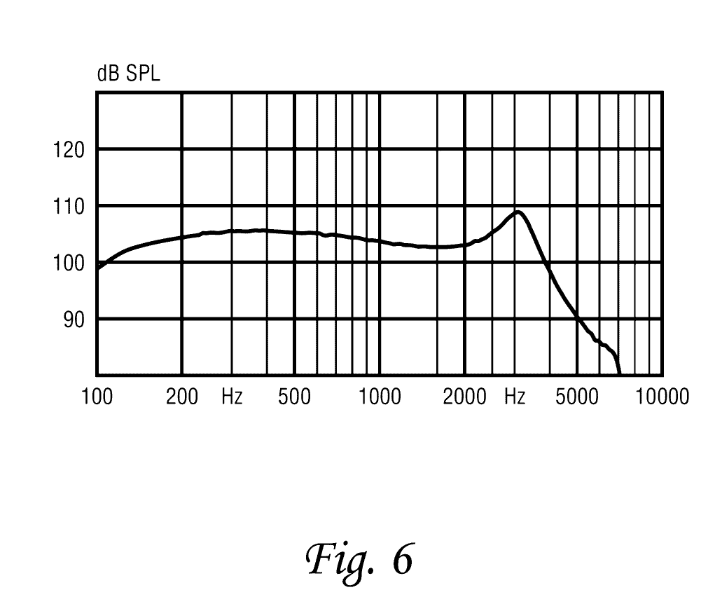

FIG. 6 is a diagram illustrating an example of the output sound pressure level (OSPL) of a conventional hearing aid. As this example illustrates, this conventional hearing aid produces an output of greater than 100 db SPL from approximately 100 Hz to just under 4 kHz, and rolls off from those levels above 4 kHz. This examples falls to 80 db SPL at about 7 kHz. This curve is for a conventional hearing aid measured according to ANSI 53.22 (2003) and S3.7 (1995), IEC 60118-7 (2005) and IEC 60318-5 (2006).

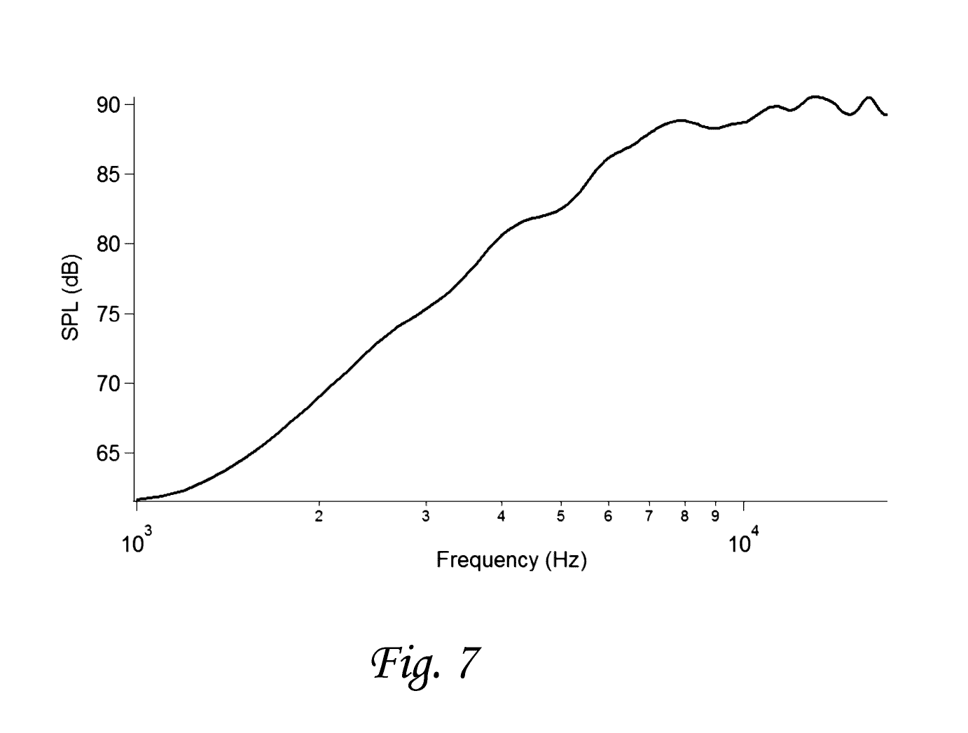

FIG. 7 is a diagram illustrating an example of the output sound pressure level (OSPL) of an ultrasonic emitter made in accordance with embodiments of the techniques disclosed herein. As can be seen in this example, the emitter provides 80 db SPL at approximately 4 kHz and approximately 90 db SPL at 10 kHz. As this curve illustrates in comparison to the curve shown in FIG. 6, ultrasonic emitters can be utilized that are better able to reproduce the high frequencies of the audio spectrum than are conventional audio hearing aids. Not only does this contribute to the superior results obtained in the aforementioned audiology tests, but the inventors believe that it also contributes to superior results for tinnitus masking systems.

In contrast to conventional hearing aids, ultrasonic audio systems in accordance with the systems and methods described herein can be configured to provide audio signals at 10 to 12 kHz or higher whether in freestanding ultrasonic emitters, headphone emitters or in-ear emitters. Such systems can provide clinically useful audio content above 4 kHz while delivering significantly lower sound pressure levels such as, for example, 60, 70, or 80 dB.

In some patients, the tinnitus symptoms are low pitched and can be difficult to treat with a conventional hearing aid in those subjects with hearing loss. Conventional hearing aids are also generally limited in producing low frequencies, whereas the ultrasound earpieces described herein have demonstrated the ability to deliver low frequencies. Accordingly, systems and methods can be implemented to deliver low frequencies to the listener for treatment of tinnitus symptoms. For in-ear applications, in-ear ultrasonic audio systems such as those described herein can provide low-frequency response that is superior to conventional hearing aids. For example, in ear ultrasonic audio systems can provide a low-frequency rolloff point below 500 Hz. As another example, an in-ear ultrasonic audio system can provide a low-frequency rolloff point below 400 Hz. As yet another example, an in-ear ultrasonic audio system can provide a low-frequency rolloff point below 300 Hz. As still a further example, an in ear ultrasonic audio system can be configured to provide a low-frequency rolloff point below 35 or 40 Hz. as yet a further example, an in ear ultrasonic audio system can be configured to provide a low-frequency rolloff point at 30 Hz. In some embodiments, the rolloff point is a frequency at which the response of the audio device is reduced by a determined amount (e.g., 3 dB). This is also sometimes called the cutoff frequency or, in the case of a 3 dB rolloff, the half-power point.

With the ability for the ultrasonic audio systems described herein to produce audio signals with a rolloff point as low as 30-40 Hz on the low end and as high as 10 to 12 kHz or higher on the high end, such ultrasonic audio systems can provide audio content over a broader and more useful frequency spectrum than can conventional hearing aids. Accordingly, in some embodiments, ultrasonic audio systems in accordance with the technologies described herein can deliver audio content with an audio frequency response greater than 400 or 500 Hz to 10 kHz. Indeed, some systems can provide a frequency response as wide as 30 Hz-12 kHz.

This can aid not only with hearing loss (especially at the high frequencies) but also with tinnitus treatment. As noted above, the systems and methods described herein provided a drastic improvement in speech understanding for a test sample of patients with mild to severe hearing loss. It is believed that the ability of the systems and methods described herein to deliver a broader spectrum frequency response without a high-frequency drop off contributes to this improved performance. Indeed, it has been noted by recognized audiologists and speech pathologists that most of the speech sounds that contribute to speech intelligibility are dominated by high-frequency speech components. See, for example, “The Effect of Stimulus Bandwidth on Perception of Fricative /s/ among Individuals with Different Degrees of Sensorineural Hearing Loss components” by Yadav, et. al., published in the Theory and Practice in Language Studies, Vol. 1, No. 12, pp. 1679-1687, December 2011 (ISSN 1799-2591). Accordingly, the ability to deliver high frequencies is believed to dramatically improve the performance of the ultrasonic audio systems described herein as compared to conventional hearing aids or assistive listening devices.

In accordance some embodiments, hybrid emitters and/or a plurality of emitters can be utilized. For example, in one embodiment, an in-ear ultrasonic transducer device may be operatively combined with a conventional hearing assistive device. That is, the conventional hearing assistive device may be operative between some range(s), e.g., for signals between approximately 500 Hz and 8 KHz (commensurate with conventional hearing assistive device operating limits). The in-ear ultrasonic transducer device may be operative for signals, e.g., less than 500 Hz down to 20 Hz and/or greater than 8 Khz up to 20 KHz (covering frequencies the conventional hearing assistive device is incapable of handling). In accordance with another embodiment, an in-ear transducer device may be configured/partitioned such that audio within one range of frequencies (e.g., 500 Hz-8 KHz) is transmitted conventionally, while within one or more other range(s) of frequencies (e.g., less than 500 Hz-20 Hz and/or greater than 8 Khz-20 KHz) HSS/ultrasound may be utilized.

Additionally, in ear or headphone configurations can be provided with an open or vented design, which allows other sounds to reach the listener's ear. Accordingly, a listener can use the tinnitus treatment system in such embodiments while remaining able to respond to external audible stimuli. As such, in some embodiments, the user can conduct activities such as drive a car, work, listen to music or other audio from an external source, participate in conversations with others, and so on, while undergoing the treatment.

In various embodiments, ultrasonic audio treatment can be delivered through the air or by ultrasonic bone conduction to provide tinnitus masking and the devices described herein can be configured to provide upper frequency audio and any variety of ultrasonic frequencies (singular or mixed) as therapy. In some embodiments, the ultrasonic system can deliver the therapy without interfering with normal audio in the environment.

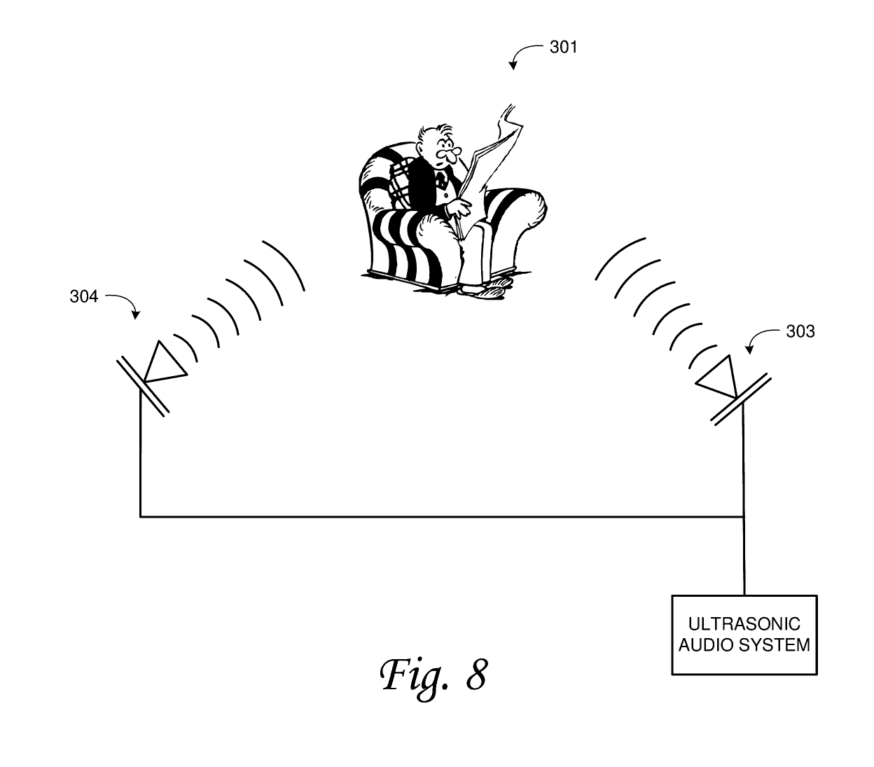

FIG. 8 is a diagram illustrating an example configuration of the tinnitus therapy system in accordance with one embodiment of the systems and methods described herein. In this example, a pair of ultrasonic emitters 303, 304 are provided and directed at a listener-subject 301. In accordance with the exemplary processes and techniques set forth herein, the listener 301 can choose the audio therapy content that she or he wishes to hear or that is prescribed by an audiologist or other hearing health professional. In some cases, the listener 301 might choose from among a variety of audio content available or prescribed. In other cases, the listeners health care professional may have prescribed a specific group or particular class of audio as therapy content for the listener 301. In these other cases, the listener 301 chooses from among the available audio content.

The listener may direct the ultrasonic emitters toward a listening position such that the ultrasonic signals are directed at the listener. Accordingly, the listener will be able to hear the audio content modulated onto the ultrasonic carrier. Because of the directional nature of the ultrasonic signals, other people in the vicinity of the listener but not in the path of the ultrasonic signals (or their reflections, if any) will not be able to hear the ultrasonic content. Accordingly, the listener will be able to enjoy the benefits of the ultrasonic signals or the audio-modulated therefrom to relieve the tinnitus symptoms, while not disturbing others in the area. This can be ideal for home or work environments where there may be others who do not want to listen to the audio content directed at listener.

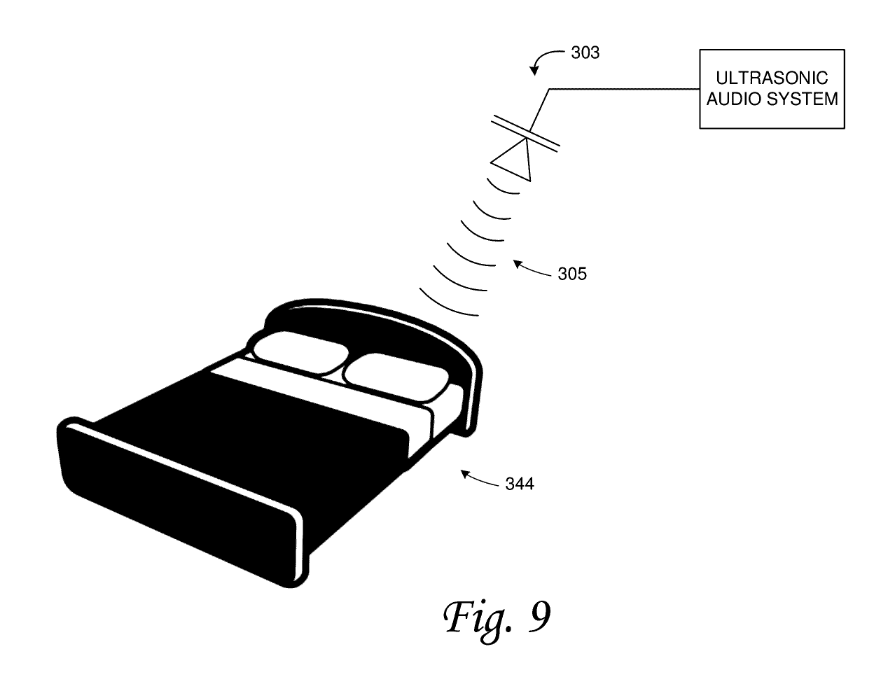

As a further example, this can be ideal at nighttime where the user can have audio-modulated or unmodulated ultrasonic signals directed toward himself or herself without disturbing a sleeping partner or spouse. See, FIG. 9, for example, which illustrates an ultrasonic emitter 303 mounted so as to direct the ultrasonic signal 305 toward one side of the bed 344 (e.g., mounted to the ceiling, wall, headboard, bedside stand, etc.).

The system can be used in environments where there are other listeners who would also like to hear the audio content directed at the listener. In such environments, the system can be configured to provide a larger listening area. For example, curved emitters with convex emitting surfaces can be used to provide a larger listening area. As another example, multiple emitters can be provided to direct the ultrasonic signal toward multiple listeners either directly or indirectly by reflecting the signal off of walls, windows, or other surfaces in the listening area. As yet another example, ultrasonic emitters can be used in combination with conventional audio speakers to provide a broader listening area. For example, the conventional audio speakers can be included to provide the audio content to listeners in the room across a broad listening area, while the ultrasonic emitters can be used to provide the same audio content modulated onto an ultrasonic carrier targeted to the intended listener. Alternatively conventional audio may be provided by the conventional speakers to listeners in the room and tinnitus therapy may be delivered to the listener through ultrasonic emitters. In these ways, the intended listener can reap the benefits of the ultrasonic signal or the audio modulated ultrasonic signal while other listeners can also enjoy the audio content.

While the components of the system discussed above are generally positioned in an area in which the subject may move about (e.g., an office space, home, public venue, etc.), in some embodiments the ultrasonic emitter(s) can be positioned very near, at, or in an ear(s) of the subject and demodulation of an audio-encoded ultrasonic carrier signal can be accomplished within the listener's ear pathway(s). Accordingly, in yet another embodiment, the ultrasonic emitter or emitters can be positioned in headphones or on an earpiece or pair of earpieces or a headset, such that the emitters themselves can be placed in close proximity to the listener's ears. As another example, the earpiece or earpieces could include earpiece mountings similar to those used for conventional hearing aids or assistive listening devices. The earpiece can include openings that allow ambient (audible) sound waves to pass through the earpiece when the earpiece is positioned over or within a user's ear allowing tinnitus therapy content without blocking normal room audio. Also the ultrasonic emitter may be positioned very near, at or in the ear of the subject using a small ultrasonic transducer.

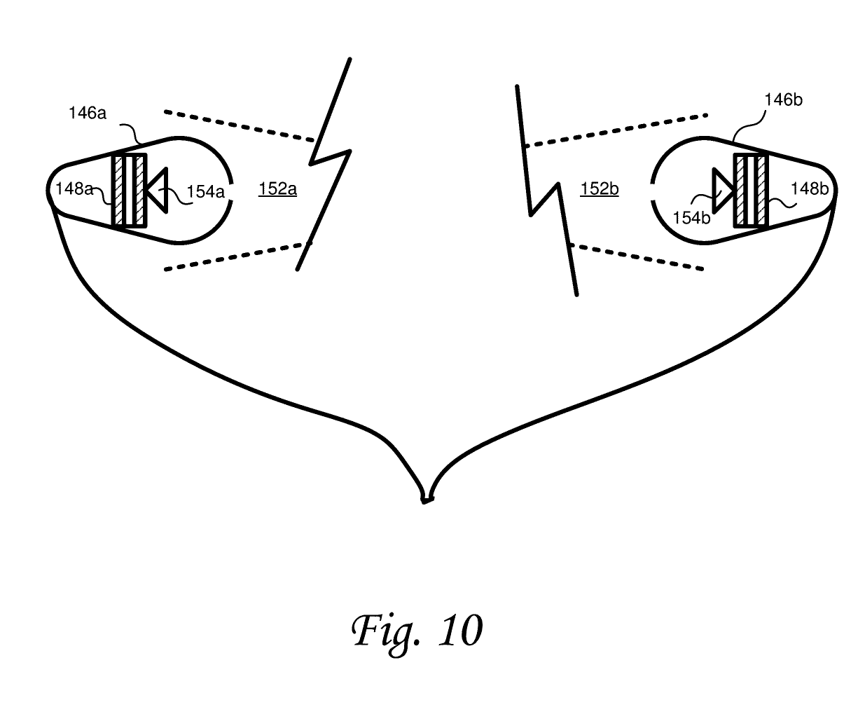

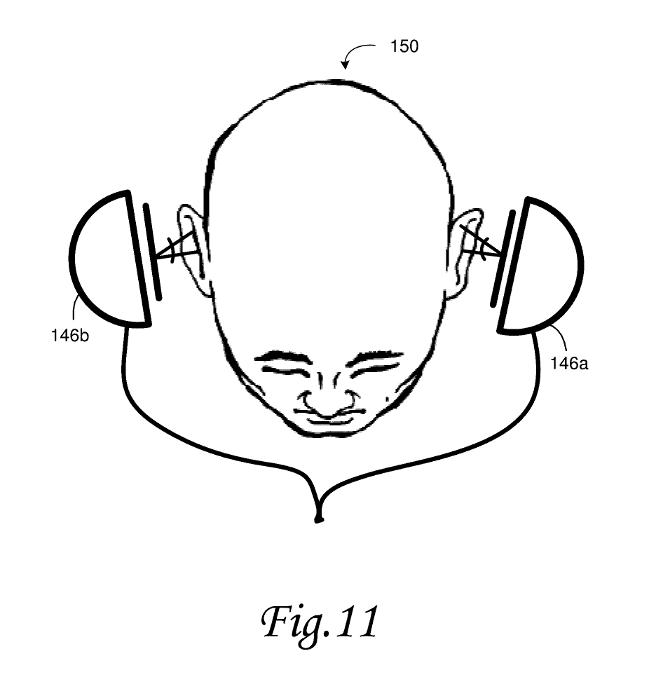

Accordingly, in some embodiments, ultrasonic emitters can be implemented in headphones or earbuds. FIG. 10 illustrates a cutaway view of an example earbud configuration, while FIG. 11 illustrates a view of an example headphone configuration. With reference to FIG. 10, in this example housings/enclosures 146a, 146b are illustrated as containing bimorph ultrasonic emitters 148a and 148b. Implemented in conjunction with bimorph ultrasonic emitters 148a and 148b are impedance matching cones 154a, 154b, respectively. Impedance matching cones 154a and 154b can be configured to match the impedance within ear canals 152a and 152b, respectively.

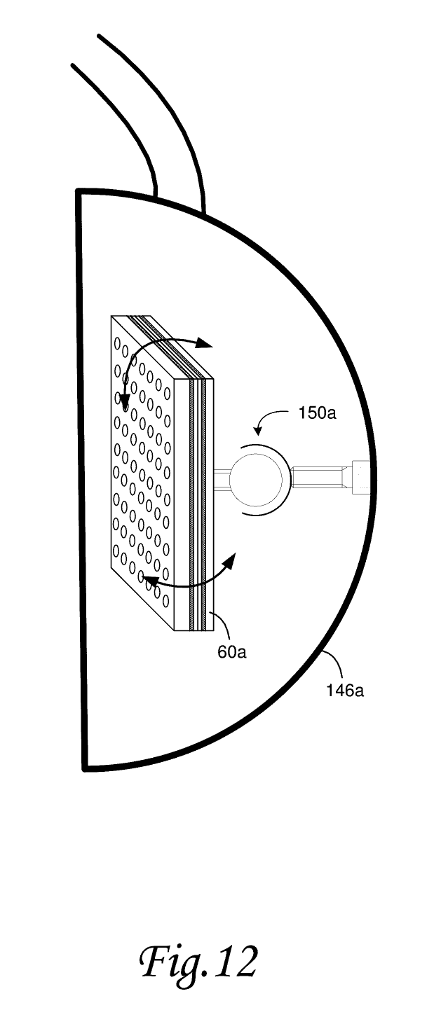

FIG. 11 illustrates left and right portions of ultrasonic headphone system 146a, 146b directed to left and right ears of user 150. The left and right portions 146a, 146b can include an ultrasonic emitter mounted in each earpiece, an example of which is shown in FIG. 12. The left and right portions of ultrasonic headphone system 146a, 146b (i.e., the earpieces) can be implemented as on-the-ear or over-the-ear earpieces, and can be adjustable relative to the ears of the listener. Accordingly, ultrasonic emitters may be configured to be adjustable in one or more directions simultaneously, e.g., horizontally, vertically, pitched, rolled, etc. and/or mounted in any desired position or orientation.

In accordance with some embodiments, ultrasonic emitters may be mounted in a fixed position and orientation. For example, headphones can be configured with the emitters oriented in such a way that the emitted ultrasonic signal travels toward the ear canal of the listener. The position or angle of direction in which ultrasonic emitters face relative to the ears of user 150 can vary, depending on the size of the earphone housings and depth of placement of the emitters therein. For example, in some embodiments the emitters are angled approximately 20 degrees towards the front of the head of user 150 in order to achieve an optimal direction of ultrasonic wave transmission into the ear canals. In other embodiments, other mounting angles can be used. As a further example, angles in the range of 5-30 degrees can be used.

In order to optimize directionality of the ultrasonic waves emitted from the ultrasonic emitter, the ultrasonic emitter can be implemented on an adjustable base or enclosure. This can allow the emitter to be pivoted or oriented within the headphone housing to ‘aim’ the emitter, and the emitted audio-encoded ultrasonic signal, in a desired direction to improve the listener's ability to hear the generated audio. For example, the ultrasonic emitter may be mounted onto a ball joint that can be rotated or pivoted within a socket in each housing/enclosure of an in-ear headphone ultrasonic transducer system. In other embodiments, any type of adjustable mechanism may be used to allow for adjusting and setting the ultrasonic emitter in a desired position and orientation relative to the ears/ear canals of a user. Accordingly, the ultrasonic emitter may be configured to be adjustable in one or more directions simultaneously, e.g., horizontally, vertically, pitched, rolled, etc. and/or mounted in any desired position or orientation.

FIG. 12 is a cutaway diagram of ultrasonic headphone housing 146a in accordance with one embodiment. As described above, one mechanism that may be utilized to orient an emitter in a desired position, e.g., relative to a user's ear/ear canal, is a ball and socket joint. FIG. 12 illustrates that emitter 60a may be mounted to the “ball” portion of ball and socket joint 150a, which may be received in the “socket” portion of ball and socket joint 150a. Accordingly, a frontal plane of emitter 60a may be rotatably positioned and/or fixed in a desired position, e.g., at an angle 20 degrees towards the rear of the user's ear/ear canal. It should be noted that ball and socket joint 150a may be utilized, in accordance with some embodiments to orient and thereafter maintain emitter 60a in a desired position, or alternatively can be made accessible to the user to allow for adjustments to be made by the user. The friction between the ball and socket can be sufficient to allow the emitter to be held in a selected orientation via a friction. In accordance with another example, the ultrasonic emitter may be mounted on a rack and pinion arrangement or ratcheting-adjustment mechanism.

In further embodiments, the adjustment mechanism to allow the orientation of the emitter to be changed can be controlled electronically using external signaling. Accordingly, the sound qualities delivered to the listener can be altered by adjusting the positioning and orientation of the emitters during the listening event. For example, the audio signal delivered by the audio source may be encoded with additional information they can be used to alter the position or orientation of the emitters. As a further example, in a gaming environment signals to control the position and orientation of the emitter can be generated to adjust the emitter based on occurrences in the game. Similar techniques can be used to adjust the audio experience for television or movie program content to provide a more spatial effect using information encoded on the signal line delivered to the headphones. Accordingly, in such embodiments, motorized mounts can be provided to adjust the position or orientation of the emitters based on these encoded signals.

Various further techniques can be combined with the systems and methods described herein for personalized tinnitus therapy devices. For example, in some embodiments a tinnitus treatment profile of a listener can be determined and established for that listener. The tinnitus treatment profile can identify therapy parameters, or operating parameters for the treatment device, such as ultrasonic audio system parameters such as carrier frequency and signal strength for an audio modulated ultrasonic carrier signal. Therapy parameters identified can also include audio content to be delivered to the listener to at least partially mask or otherwise alleviate the listener's tinnitus symptoms and offer the benefit of residual inhibition. The audio content can be identified by particular pieces of audio content or by a group or class of audio content. For example, the treatment profile for patient can identify particular pieces of audio content suitable for the patient based on his or her symptoms and reactions to the content. Likewise, classes of audio content can be identified such as, for example, tones (single or multi tones), tones at a particular frequency or within particular frequency ranges, speech or music, music of a particular type or genre, white noise, pink noise, red noise, brown noise sound pressure level of the delivered therapy, and so on.

Because different listeners may respond differently to the ultrasonic signals, a tinnitus treatment profile can identify those ultrasonic audio system parameters that provide good results for the listener. In various embodiments, the tinnitus treatment profile can be determined in conjunction with a health-care professional such as, for example, through an examination and evaluation process by the health-care professional. For example, ultrasonic audio system parameters (including the audio content) best suited for treating the patient's particular tinnitus symptoms can be determined by a clinical professional.

In other embodiments, the health-care professional for the user can conduct a trial and error process in which various parameters are varied to optimize the results. In yet other embodiments, the tinnitus treatment profile can be determined by the listener through adjustments to the ultrasonic audio system parameters (including the audio content). In other embodiments, the signal parameter of the ultrasonic audio system can be configured based on a hearing response profile of an intended tinnitus patient. Examples of employing a hearing response profile are disclosed in U.S. Pat. No. 8,929,575, which is incorporated by reference herein in its entirety. The system, in yet other embodiments, can be configured such that the listener can make adjustments to the various parameters, such as frequency response, to determine desirable settings to mask or alleviate the tinnitus symptoms. Because the symptoms may vary for a particular listener throughout the day, adjustable settings can be desirable to allow the listener to tailor the system to meet his or her needs. Alternatively, the efficacy of the treatment may vary based on environment in which the treatment is being performed. Ambient noise levels in the environment, types or amounts of background noise, acoustical characteristics of the room (including surfaces and furnishings), and other factors can influence the type of treatment in the system parameters desired.

Additionally, a system memory can be provided to store a number of preset configurations. The preset configurations can be established by the health care professional or the listener so that predetermined parameter settings can be selected relatively quickly. For example, a user interface can be provided with preset buttons to enable preset selection.

There are a variety of content items that can be used for tinnitus masking or treatment including, for example, music content, spoken content, tones, white noise, pink noise, red noise, brown noise or a combination thereof. Tones can include, for example, single-frequency tones, time-varying tones, chords or other multi-tone tones, and so on. Music content can include any of a number of music genres or types. Any of these or similar content will benefit from the clarity of ultrasonic audio delivery and the ability to deliver higher frequency content to those with mild to severe hearing loss as compared to normal hearing aids. Likewise, as noted above, delivery of ultrasonic wave patterns can be delivered without audio content to provide therapy through delivery mechanisms such as those described herein.

It should be noted that the use of a parametric ultrasonic wave or a HSS signal in accordance with various embodiments holds particular advantages over conventional tinnitus masking devices employing conventional audio speakers, headphones, earbuds or similar audio reproduction elements. That is, various embodiments, through the use of ultrasonic signal delivery, may be configured to provide a perfect or at least near-perfect transient response, which can improve clarity, as opposed to conventional audio systems that can experience various types and/or varying amounts of distortion due to, e.g., the mass and/or resonance of drivers, enclosures, delay, etc.

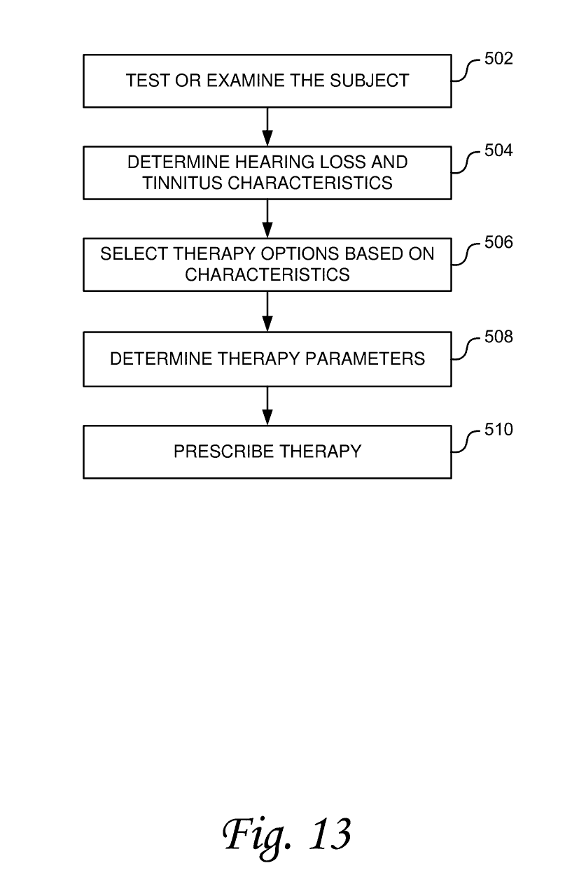

The ultrasonic treatment options can be chosen according to a number of different criteria. FIG. 13 is a diagram illustrating an example process for determining the ultrasonic therapy in accordance with one embodiment of the systems and methods disclosed herein. Referring now to FIG. 13, at operations 502 and 504 the clinician or other health care professional tests or examines the subject to determine the nature and characteristics of the tinnitus symptoms and if applicable the nature of any hearing loss. In these operations, the practitioner can interview the subject in an attempt to determine the cause and nature of the tinnitus symptoms. For example, the practitioner may inquire into items such as the volume of the tinnitus symptoms, the type of sound detected (e.g., high-pitched squeal, hissing, buzzing, etc.), times of day or locations at which the symptoms occur, and so on. The practitioner may also administer a hearing test to determine if hearing loss at some or all of the normal audible frequency range has occurred, and if so, to what extent.

At operation 506, the practitioner can select a therapy or therapy options based on the characteristics of the symptoms determined at steps 502 and 504. For example, the practitioner may determine whether ultrasonic therapy is appropriate, and whether it should include audio-modulated or unmodulated ultrasonic signals. Where audio-modulated ultrasonic signals are chosen, the practitioner may determine the type of audio content to be delivered to the listener, or whether any form of audio content generally would be suitable. For example, the practitioner may determine that audio content with an emphasis on high-frequency content may be more suitable for some subjects, whereas any form of audio content in general may be suitable for other subjects.

Also, as noted above, a tinnitus treatment profile of a listener can be determined, and audio content (or other ultrasonic audio parameters) can be selected or adjusted to at least partially compensate for the listener's tinnitus treatment profile. In still further embodiments, the practitioner may choose from a variety of commercial masking therapy content available.

As a further example, the practitioner may determine that simply providing a masking sound using ultrasonic audio emitters is a recommended course of therapy. As another example, the practitioner may determine that high-frequency hearing loss contributes to the symptoms, and that delivering audio content to the subject that compensates for the high-frequency hearing loss can help to address a cause of the symptoms, thereby mitigating the symptoms themselves. For example, in situations where the cause of the symptoms is associated with loss of hearing at the high frequencies, providing the subject's brain with the missing information (high-frequency audio the subject can hear) may be sufficient to alleviate some tinnitus symptoms. In this and other situations, simply providing audio content to the listener can be helpful to mask the symptoms. With some subjects, the symptoms may be mild enough that simply providing background audio to listener is sufficient to mask the symptoms or to simply distract the subject from focusing on the symptom.

At operation 508, the practitioner can determine ultrasonic audio system parameters for the therapy. The parameters can include, for example, the frequency and duration of therapy sessions, the volume or signal strength at which the audio content or ultrasonic signals are delivered, the ultrasonic carrier frequency (whether or not modulated with audio content) the type of audio content modulated onto the ultrasonic carrier, the audio content frequencies, types of noise or sound stimuli mixed with actual audio content for delivery, and so on. The parameters can be determined and recommended by the practitioner, and adjusted based on results obtained with the subject. For example, the practitioner and the subject can experiment with different audio content in different frequency and other settings to achieve therapy characteristics and parameters that work well with the subject's condition. These therapy characteristics can be part of a tinnitus treatment profile for the listener.

At operation 510, the practitioner prescribes a therapy to the subject. The practitioner may deliver particular audio content to the subject, or may describe to the subject the type of audio content that would be suitable for the prescribed therapy. With this information, the subject can obtain an ultrasonic audio system and recommended content, and follow the prescribed routine.

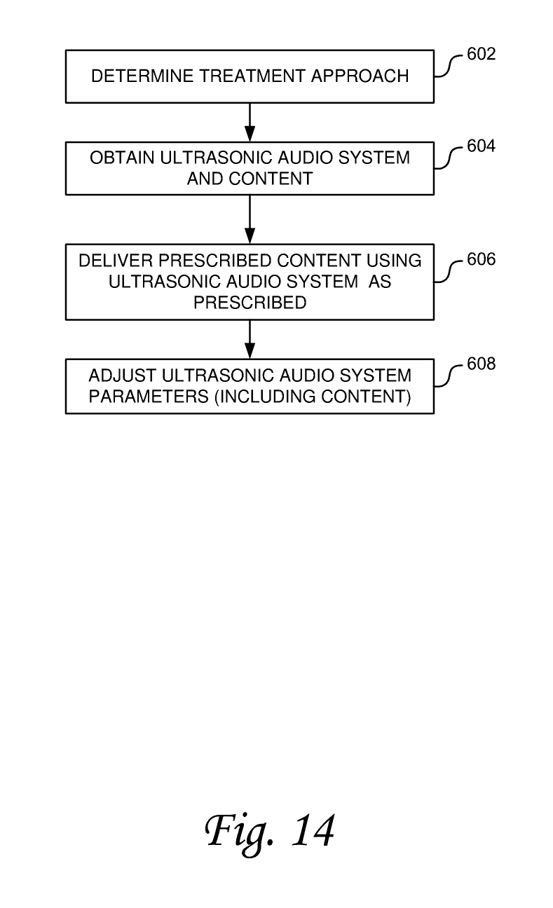

FIG. 14 is a diagram illustrating an example process for administering the treatment in accordance with one embodiment of the technology described herein. Referring now to FIG. 14, at operation 602, the treatment approach is determined. For example, in various embodiments, the treatment approach can be determined as described above with reference to FIG. 13. In other embodiments, other techniques can be used to determine the treatment approach including self-help techniques.

At operation 604, the subject obtains an ultrasonic audio system by which to administer the treatment. The subject can also obtain audio content such as, for example, where it is determined that audio content is part of the treatment approach. With the ultrasonic audio system in place, the subject delivers the treatment in accordance with the determined treatment approach. As noted above, in various embodiments, the treatment may entail delivering an unmodulated ultrasonic carrier to the subject according to prescribed parameters. Also, in further embodiments, the treatment may entail delivering audio content via the ultrasonic audio system, which can also be done using prescribed parameters. Accordingly, at operation 608 the subject can adjust the ultrasonic audio system parameters (including the audio content, if any) to improve or optimize the treatment. The system can be configured to allow manual adjustment by the user so the user can change the settings (or the content) to best suit the user's symptoms or based on a particular environment in which treatment is being administered.

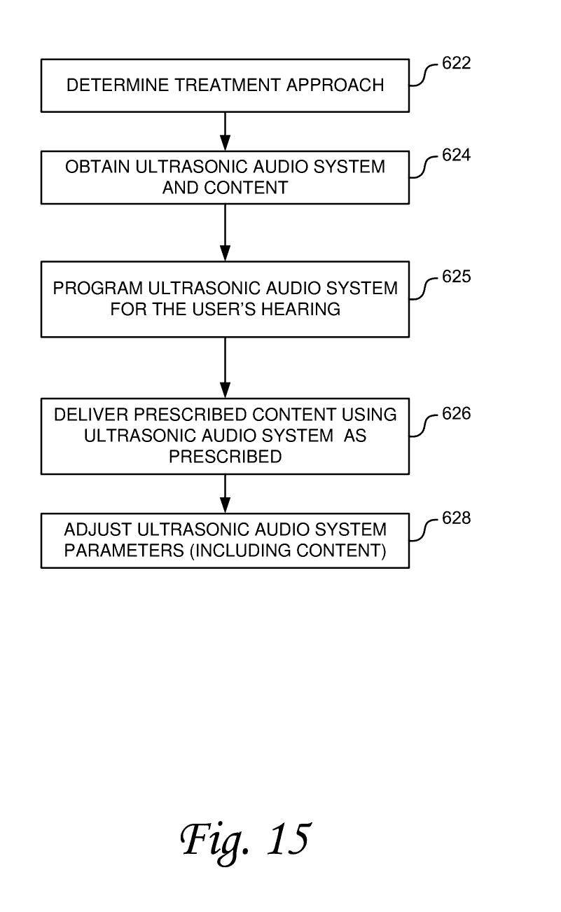

In further embodiments, determination of the treatment approach can further include consideration of the subject's hearing response profile. With a determined hearing response profile, the ultrasonic audio system can be adjusted to account for the subject's particular hearing loss. FIG. 15 is a diagram illustrating an example process for administering the treatment in accordance with an embodiment in which the subject's hearing response profile is also determined. FIG. 15 includes the steps previously outlined above in FIG. 14, in which steps 622, 624, 626 and 628 correspond to steps 602, 604, 606, and 608, respectively. The example of FIG. 15 further includes the additional step 625 of the healthcare professional configuring the signal parameters of the ultrasonic audio system based on a hearing response profile. The tuning based on the hearing response profile can adjust the equalization of the audio system to provide frequency-dependent gain (e.g, high-frequency or low-frequency gain) as appropriate based on the patient's hearing response profile. This may involve, for example, providing increased output at various frequency ranges including higher frequency ranges where hearing loss often occurs. As noted above, using an ultrasonic audio system for delivery of the treatment can be beneficial over conventional audio hearing aids as typical hearing aids have difficultly delivering high-frequency content, especially to persons with mild to severe hearing loss. The adjustments in step 625 for the patient's hearing response profile can be in addition to adjusting the tinnitus treatment parameters of the system based on the tinnitus treatment profile and the preferences of the intended tinnitus patient.

As noted above, due to the directional nature of ultrasonic signals, the listener can enjoy the therapy in a variety of public or private settings such as, for example, his or her home or office, while on public transportation, in public places, and so on.

As noted above, the inventors have discovered that patients with mild to severe hearing loss experience a significant improvement in sound clarity when listening to speech and other audio content through ultrasonic emitter systems such as those described above, as compared to conventional audio speakers. This is true even in the presence of background noise. Accordingly, ultrasonic audio systems such as those described herein can be used as a hearing aid or other assistive listening device. For example, in some embodiments, a microphone can be provided to capture sounds and provide those sounds as input (or audio content) to an ultrasonic audio system. As a further example, a microphone can be included to be worn on the listener's lapel or it can be integrated into a hearing aid or other assistive listening device to be worn on or within the listener's ear. The microphone can be configured to pick up sounds such as speech, music, or other sounds and provide those sounds to an ultrasonic audio system, such as, for example, that described above with respect to FIGS. 1 and 2. The ultrasonic audio system modulates the captured sound energy onto an ultrasonic carrier, amplifies the signal, and launches the signal by the emitters, which are directed toward the listener's ear. Accordingly, such a device can be worn anytime by the listener to improve his or her hearing as well as to alleviate tinnitus symptoms. In circumstances where the tinnitus is brought about by hearing loss, such a device, when used as an hearing aid or assistive listening device with the improved sound clarity levels exhibited during testing, may not only enable the listener to hear better, but may for that reason, also alleviate the tinnitus symptoms simultaneously by delivering clear masking content. Accordingly, utilizing the described ultrasonic audio systems as a hearing aid or assistive listening device may allow the normal everyday sounds heard by the listener to provide the audio content that alleviates the tinnitus.

The examples described above with reference to FIGS. 13, 14 and 15 rely on a physician, audiologist or other health care practitioner to define the parameters of the treatment. In the above-described and in other embodiments, the patient can be given a custom audio player or application for a portable device (e.g., for a smartphone, tablet, mp3 player etc.) to allow the patient to adjust the therapy parameters to his or her liking. Accordingly, in some embodiments the patient selects and customizes some or all of the tinnitus treatment parameters. In further embodiments, this patient-customizable audio player can further be set or adjusted by the health care practitioner for determined tinnitus therapy parameters and the patient's hearing response profile and later adjusted or customized by the doctor or patient.

Whether doctor or patient adjustable (or both), the audio player (which can be an application as described above) can further be configured to allow a particular stimulus or stimuli to be selected, adjusted and added or mixed in with audio content so that the audio player delivers the audio content combined with selected audio stimulus or stimuli. Stimuli can include, for example, tones, nature sounds, white noise, pink noise, brown noise, or other sounds. These stimuli can be constant or they can vary in either or both frequency and amplitude, and can be selected based on the relief provided to the tinnitus symptoms.

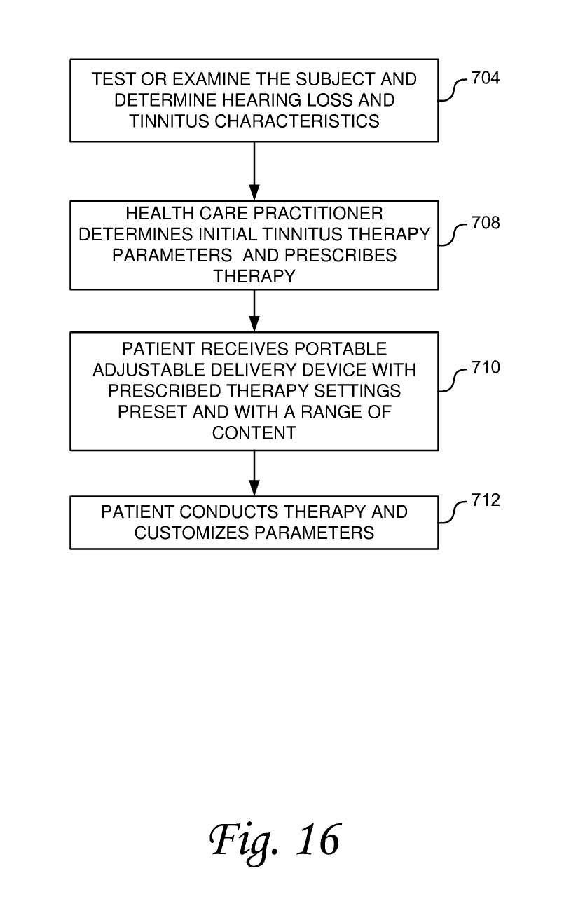

An example of configuring and adjusting a custom audio player is shown at FIG. 16. In this example, the clinician or other practitioner tests or examines the subject to determine the nature and characteristics of the tinnitus symptoms and if applicable the nature of any hearing loss. This is shown at 704. In these operations, the practitioner can interview the subject and work to determine the cause and nature of the tinnitus symptoms. For example, the practitioner may inquire into items such as the volume of the tinnitus symptoms, the type of sound detected (e.g., high-pitched squeal, hissing, buzzing, etc.), frequency of the symptoms, times of day or locations at which the symptoms occur, severity of the symptoms, and so on.

At operation 708, based on the information determined in the patient evaluation, the practitioner determines the therapy parameters under which the treatment will be administered. This can include, for example, various ultrasonic audio system parameters as discussed in various embodiments, above. The practitioner configures an ultrasonic audio system to deliver therapy according to the determined parameters.

The practitioner may also administer a hearing test to determine if hearing loss at some or all of the normal audible frequency range has occurred, and if so, to what extent. If there is hearing loss at some or all of the normal audible frequency range, the practitioner may also program or tune the ultrasonic audio system to account for the patient's hearing loss (including particular hearing loss characteristics such as his/her hearing response profile as illustrated in FIG. 15 and described above). The tuning can be configured to provide frequency dependent gain (e.g, high-frequency or low-frequency gain) as appropriate based on the patient's hearing loss profile.

At operation 710 the user is provided a customizable audio player or application for a portable device that allows the patient to customize the tinnitus therapy parameters him- or herself. For example, a stand-alone audio player or an application (e.g., for a portable device such as a smart phone, tablet, computer, mp3 player or other like device) can be provided to the patient and can include the ability to select custom content for the treatment, use pre-loaded content (e.g., music, voice, tones, nature sounds, white noise, pink noise, brown noise, or other sounds), use patient loaded content, or a combination of the foregoing.

At operation 712, the patient can adjust one or more of the ultrasonic audio system parameters to tailor the treatment based on changes in symptoms, conditions in the listening environment, personal preferences, efficacy of the treatment, and so on. This can include adjusting the equalization of the audio delivery, adjusting or selecting the audio to modulate onto the carrier, adjusting the delivered sound pressure level, and so on. The audio can be audio content (e.g., music, voice, nature sounds, noise sounds, tones and other sounds), stimuli (e.g., tones, white noise, pink noise, brown noise, or other sounds), or a combination of the foregoing. Additionally, embodiments can be configured in which users (patient or health care provider) can mix or combine one or more stimuli with audio content, and can further adjust the level of mixing. While in some embodiments, more traditional audio content such as music or speech is mixed with the stimulus such as a tone or noise, any audio content including tones, noises and other sounds can be mixed with any stimulus or stimuli.

This custom adjustment of these various parameters allows the patient to tailor the tinnitus treatment parameters to his or her liking, and may further allow adjustment to select or customize different therapies for different environments. For example, a user may determine that certain parameters yield better results when he or she is in bed or sleeping, while other parameters yield better results when he or she is in a noisy environment, and so on. The customizable audio player can include a user interface to allow adjustment of these parameters. Additionally prescribed parameter settings and custom parameter settings can be programmed and stored into the customizable audio player to allow recall of preferred settings.

The adjustment and selection of treatment parameters described above at operation can be reviewed by a health care practitioner, and from this information, the practitioner can recommend revised therapy parameters based on the tinnitus and patient characteristics determined at operation 704 and based on the user preferences determined at operation 712. In some embodiments, the customizable audio player can include memory for storing a history of user settings so that this history can be reviewed by, for example, a health care practitioner.

As the above examples illustrate, the systems and methods disclosed herein can be configured to provide a personal, user-driven tinnitus therapy system that allows the user to use prescribed treatment parameters and to customize the treatment parameters to suit his or her symptoms, tastes, or preferences, or to alter the parameters based on the treatment environment, the time of day and so on. The system can also be configured with preset stored parameters and to store user preferences so preset sets of parameters can be recalled for treatment. Moreover, this can allow multiple levels of personalization. That is a first level can be personalization based on a prescribed set of parameters, which may also include parameters set based on the patient's personal hearing loss (e.g., his or her hearing-loss profile); and the second level can include adjustments to those parameters made by the patient (or the physician) while operating the system. Thus the user can adjust and improve the parameter settings based on preferences, environment, symptoms or other factors.

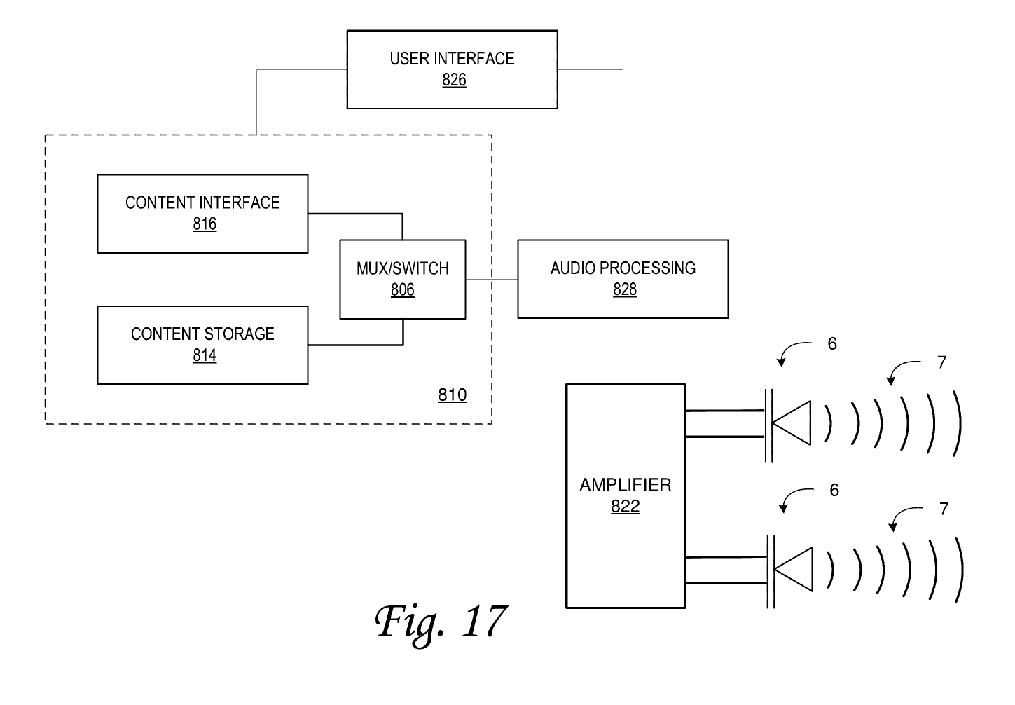

As described above, in various embodiments the user is provided with an ultrasonic audio system or device that can be used to administer the ultrasonic tinnitus therapy. Such a device can take a number of different configurations, and can be implemented using hardware, software, or a combination thereof. FIG. 17 is a diagram illustrating an example of such an ultrasonic audio system. With reference now to FIG. 17, in this example application, the example ultrasonic audio system includes a user interface 826 that can be configured to allow adjustment of the tinnitus therapy parameters as discussed in various embodiments above. Depending on the implementation of the ultrasonic audio system, user interface 826 can include, for example, a touchscreen interface; buttons, switches, slide controls, voice commands and the like; or other mechanisms to accept user input. The user interface can also include a communications interface to allow the ultrasonic audio system to receive parameter-setting information from external devices electronically. For example, the ultrasonic audio system can include a wired or wireless communication interface to allow the ultrasonic audio system to be coupled with and communicate with external devices. Through this interface the ultrasonic audio system can receive send updates or instructions from external devices to adjust the ultrasonic audio system parameters.

In the example shown in FIG. 17, user interface 826 can be used to control content selection as well as audio processing parameters controlled by audio processing block 828. This can include, for example, equalization settings to adjust output at various frequency ranges. Also, in various embodiments user interface 826 can be configured to display current parameter settings, available parameter settings, stored settings, historic settings, available content, selected content, and so on. Although not illustrated, memory can be included to store information such as, for example, user settings, prescribed settings, favorites, and historic information.

Audio processing block 828 receives audio content from audio content block 810 and processes the received audio content to generate the desired ultrasonic signal to drive the ultrasonic emitters. Accordingly, audio processing block 828 can be configured to perform equalization, compression or expansion, filtering, modulation (onto an ultrasonic carrier), and other audio processing functions as may be desired to alter or enhance the ultrasonic signal or the content modulated thereon. As one example, audio processing block 828 can implement the components illustrated as signal processing system 10 in FIG. 2. Audio processing block can be implemented as an analog or digital block, or a combination thereof, and can be implanted using hardware, software, or a combination of hardware and software.

The ultrasonic signal generated by the audio processing block 828 is provided to an amplifier 822 for amplification and delivery to ultrasonic emitters 6. In this example, a 2-channel audio system is shown having two ultrasonic emitters 6. In other embodiments, other numbers of channels and emitters can be implemented. The emitters can be positioned at a distance from the user or they can be implemented as headphones, earphones, earbuds or in-ear transducers as described above.

As noted above, user interface 826 can also control content selection from content block 810. In this example, content block 810 includes content storage 814, a content interface 816 and a mux or switch 806. Content interface can include, for example input audio jacks and electronics to interface to and receive signals from an external audio source such as, for example, an MP3 player, a microphone, a set-top box, a streaming media source, or other external device. Content storage 814 can include, for example, any of a number of different storage media including media such as, for example, memory, a data storage device, CD, DVD, or other instrumentality for storing audio content. Content storage 814 can include internal storage into which content can be loaded (or downloaded) from a variety of external sources including from external devices, the Internet, or a communication network. Mux/Switch 806 can be provided to allow selection of the desired audio content source. Although not illustrated, additional layers of switching can be provided to allow selection from among more than two sources or to allow selection from multiple sources within content storage 814.

As noted above, the audio sources can include external audio sources such as an MP3 player, a microphone, a set-top box, a streaming media source, or other external device. Accordingly, in one embodiment, the system can be configured to operate as a hybrid listening device that allows delivery of content via an ultrasonic audio system in which the parameters of the audio system are adjusted to optimize tinnitus therapy. Accordingly, with such a hybrid device, the user can engage in activities that he or she would normally engage in (like, e.g., watch a movie or television program, listen to music, converse with others, etc.) and use the tuned ultrasonic audio system to engage in these activities. In the example of a movie or television program, the user can hook up the ultrasonic audio device to the set-top box or other audio source (e.g., via an input jack, or wirelessly) and listen to the audio soundtrack via the ultrasonic audio device. Listening to the audio soundtrack through the ultrasonic audio system can allow the user to engage in normal activities while at the same time enjoying the benefit of the tinnitus therapy. Likewise, when the tinnitus treatment system is configured as a hearing aid, the user can listen through an external microphone and the audio content from the microphone can be delivered to the listener conditioned with the tinnitus treatment parameters so that the user can engage in normal activities while at the same time enjoying the benefit of the tinnitus therapy.

The example illustrated in FIG. 17 not only illustrates an example architecture for an ultrasonic audio system. As will be appreciated by one of ordinary skill in the art after reading this description, the features and functions of the ultrasonic audio system can be implemented using alternative structures and can be implemented using hardware, software, or a combination thereof. As noted above, components of the ultrasonic audio system can be implemented as an application such as, for example, an application for a smart phone, tablet, or other like computing device. Accordingly, features like the user interface, content selection, and audio processing can be performed by an application running on a computing device.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A tinnitus treatment device, comprising:
an ultrasonic signal generator;
an amplifier coupled to the ultrasonic signal generator; and
an ultrasonic emitter including an input coupled to an output of the amplifier and being configured to output an amplified ultrasonic signal into the air;
wherein operating parameters of the tinnitus treatment device are configured in accordance with a tinnitus treatment profile determined for a patient to deliver ultrasonic tinnitus treatment signals customized to the patient.

2. The tinnitus treatment device according to claim 1, the operating parameters comprise frequency and signal strength for the ultrasonic signal.

3. The tinnitus treatment device according to claim 1, further comprising a modulator including a first input for coupling to an audio content source a second input coupled to the ultrasonic signal generator and an output coupled to the amplifier, the modulator configured to modulate determined audio content onto the ultrasonic signal to deliver selected audio content to the patient.

4. The tinnitus treatment device according to claim 3, wherein the operating parameters comprise at least one of frequency and signal strength for the modulated ultrasonic signal.

5. The tinnitus treatment device according to claim 3, wherein the audio content to be delivered to the patient comprises specific pieces of audio content suitable for the patient based on his or her symptoms and reactions to the content.

6. The tinnitus treatment device according to claim 3, wherein the audio content to be delivered to the patient comprises at least one of, tones, a tone at a particular frequency, tones within particular frequency ranges, speech, white noise, pink noise, red noise, and brown noise.

7. The tinnitus treatment device according to claim 3, wherein the operating parameters comprise, audio content to be delivered to the patient, and a stimulus mixed with the audio content.

8. The tinnitus treatment device according to claim 1, further comprising a user interface, the user interface including user input to allow adjustment of the operating parameters of the tinnitus treatment device by the patient.

9. The tinnitus treatment device according to claim 1, the ultrasonic emitter is located at a distance of greater than one foot from the patient.

10. The tinnitus treatment device according to claim 1, wherein the ultrasonic emitter comprises a pair of ultrasonic emitters configured as headphones to be worn by the patient.

11. The tinnitus treatment device according to claim 1, wherein the ultrasonic emitter comprises a pair of ultrasonic emitters configured as earbuds.

12. The tinnitus treatment device according to claim 1, wherein the ultrasonic emitter is configured as an earpiece to be worn in the ear of a patient.

13. The tinnitus treatment device according to claim 3, wherein the selected audio content delivered to the patient is delivered with an operating frequency response of 40 Hz-10 KHz.

14. The tinnitus treatment device according to claim 3, wherein the selected audio content delivered to the patient is delivered to the patient with an operating frequency response having a low-frequency cutoff below 400 Hz.

15. The tinnitus treatment device according to claim 3, wherein the selected audio content delivered to the patient is delivered to the patient with an operating frequency response having a high-frequency cutoff above between 10 kHz-12 kHz to compensate for high frequency hearing loss.

16. The tinnitus treatment device according to claim 3, wherein the audio content source is a microphone located proximal to the emitter.

17. A hybrid ultrasonic audio and tinnitus therapy device, comprising:
an audio input;
an ultrasonic signal generator;
a modulator coupled to the ultrasonic signal generator and to the audio input, the modulator configured to modulate audio content received at the audio input onto an ultrasonic carrier generated by the ultrasonic signal generator to generate an audio modulated ultrasonic signal;
an amplifier coupled to the modulator;
a user interface coupled to at least one of the audio input, ultrasonic signal generator, modulator, and amplifier to receive input and to adjust ultrasonic audio system parameters for tinnitus therapy; and
an ultrasonic emitter coupled to the audio generating apparatus and configured to output the audio modulated ultrasonic signal.

18. An ultrasonic tinnitus therapy device, comprising:
an ultrasonic signal generator;
a modulator coupled to the ultrasonic signal generator and to the audio input, the modulator configured to modulate audio content received at the audio input onto an ultrasonic carrier generated by the ultrasonic signal generator to generate an audio modulated ultrasonic signal;
an amplifier coupled to the modulator;
a user interface coupled to at least one of the audio input, ultrasonic signal generator, modulator, and amplifier to receive input and to adjust ultrasonic audio system parameters for tinnitus therapy; and
an ultrasonic emitter coupled to the audio generating apparatus and configured to output the audio modulated ultrasonic signal.

19. A method of delivering tinnitus therapy to a user using an ultrasonic audio system, comprising:
a health care practitioner determining a set of ultrasonic audio system parameters for the tinnitus therapy based on an examination of the user;
delivering the ultrasonic audio system to the user with the determined set of ultrasonic audio system parameters for the tinnitus therapy programmed into the ultrasonic audio system;
the user using the ultrasonic audio system to deliver the tinnitus therapy and the user adjusting one or more of the determined set of ultrasonic audio system parameters to customize the delivered tinnitus therapy.

20. A method of delivering tinnitus therapy to a user using an ultrasonic audio system, comprising:
a health care practitioner determining a set of ultrasonic audio system parameters for the tinnitus therapy based on an examination of the user;
configuring the ultrasonic audio system parameters of the ultrasonic audio based on the practitioner's determination;
the user using the ultrasonic audio system to deliver the tinnitus therapy in the form of an ultrasonic audio signal;
and the user adjusting one or more of the determined set of ultrasonic audio system parameters to customize the delivered tinnitus therapy.

21. The method of claim 20, further comprising:
a health care practitioner determining a hearing response profile for the user; and
further adjusting the set of ultrasonic audio system parameters for the user based on the determined hearing response profile.

22. The method of claim 20, wherein the further adjustment comprises adjusting equalization of the ultrasonic audio system to adjust the level of one or more frequency bands of the ultrasonic audio signal.
